(19) United States
(12) Patent Application Publication
(10) Pub. No.: US 2005/0032072 A1
(43) Pub. Date: Feb. 10, 2005

(54) Fragmentation and labelling with a programmable temperature control module

(75) Inventors: Curtis R. Kautzer, San Jose, CA (US); Matt M. Morenzoni, Mountain View, CA (US); Michael C. Norris, Santa Clara, CA (US)

Correspondence Address: QUINE INTELLECTUAL PROPERTY LAW GROUP, P.C., P O BOX 458, ALAMEDA, CA, 94501, US

(73) Assignee: Perlegen Sciences, Inc., Mountain View, CA (US)

(21) Appl. No.: 10/638,113

(22) Filed: Aug. 8, 2003

(51) Int. Cl. C12Q 1/68; C12M 1/34

(52) U.S. Cl. 435/6; 435/287.2

(57) ABSTRACT

This invention provides methods and systems for precisely and consistently fragmenting and labeling nucleic acid fragments. Time and temperature programmable temperature control modules are used to provide repeatable time/temperature profiles for fragmentation and labeling reactions.

### FIELD OF THE INVENTION

The present invention is in the field of precision labeling of nucleic acids with detectable markers. Systems and methods are described to fragment, label, and hybridize nucleic acids of interest using time and temperature programmable temperature control modules.

### BACKGROUND OF THE INVENTION

Pioneering techniques to detect a substance of interest are often rough and imprecise. As the techniques mature, they can become standardized and fine tuned to allow reasonable comparisons between intra-assay and interassay results. However, even with intensive technician training, and with strict adherence to a standard operating procedures, variabilities between manual assay runs can obscure differences between samples.

Consistent handling can be required in many manual assays for high sensitivity, or for resolution of small differences between assay samples. For example, in common manual assays, such as for endotoxin or blood coagulation assays, a near robotic adherence to sample handling steps is required to obtain valid results. Starting reagents must be preheated to a precise temperature. Samples, standards, controls and reagents must be placed in assay tubes in consistent order, with uniform mixing, at precise moments in time. Procedures for reading results must take place with equal consistency, e.g., with tubes removed from incubation wells in exactly the order as they were inserted and after exactly the time interval as all the other tubes. The required levels of technician training and technician discipline can be difficult to obtain, especially in the realm of high throughput analyses.

Manual fragmentation and labeling of nucleic acids, such as DNA, e.g., in the preparation of hybridization probes, can suffer from many of the difficulties common to other manual wet chemistries. In the early days of DNA hybridizations, focus of analyses was commonly on preparation of highly labeled probes, but not necessarily on preparation of probes with consistent, fragment size, labeling, or binding affinity. However, research and clinical scientists are now asking more difficult questions about the quantity or relative proportions of nucleic acid sequences. To answer these questions, more consistent nucleic acid probe labeling procedures have been devised. For example, standard procedures have been written wherein technicians are instructed to: bring all reagents, buffers and nucleic acid samples to a starting temperature in an ice bath; consistently add and mix in an endonuclease; place fragmentation tubes in warm water bath racks; remove the tubes from the water bath racks after a predetermined time and immediately placing them in a hot heat block to stop the fragmentation by denaturing the endonuclease; place the tubes back into the ice bath to provide the starting temperature for addition of a labeling enzyme; mix the tubes and place them into the warm water bath for a labeling reaction; and, stop the labeling by placing the tubes in a hot heat block at a predetermined time. By strict adherence to consistent handling techniques and tube placement times, useful hybridization probes can be obtained for quantitative studies. Yet, the precision of quantitations and the resolution of comparisons can be reduced by accumulated inconsistencies and errors inherent in manual sample handling.

However, manual techniques for detection, quantification, and frequency estimation of nucleic acid sequences remain significantly imprecise. Inconsistencies in handling, reaction temperature profiles, and timing of reaction terminations can result, e.g., in failure of an assay to detect real differences between samples. For example, nucleic acid assays can fail due to: inconsistent order of placement and removal of tubes from baths and heat blocks, differences in tube placement times between and within assays, differences in technician response times to timer alarms, differences in tube contacts and thermal conductivity according to how tubes are placed in baths and blocks, unintended handling events such as dropped tubes and “popped” tube seals, and contamination of tubes during handling. In many cases, assay throughput is significantly lowered by attempts to increase the reliability of results.

In view of the above, a need exists for methods providing labeled nucleic acid probes with more consistent binding affinity and label intensity. It would be desirable to have systems that provide more consistent probes and increased productivity in probe labeling. Consistent and efficient production of nucleic acid probes can provide benefits in high throughput screening of nucleic acids for genes associated with disease states. The present invention provides these and other features that will be apparent upon review of the following.

### SUMMARY OF THE INVENTION

The present invention includes methods and systems for consistent labeling of nucleic acids to provide, e.g., repeatable hybridizations for high resolution comparisons between target nucleic acid sequences and between results of different hybridization assays. In many embodiments of the inventions, programmable heating modules are used to provide consistent labeling reactions from one assay to the next.

Methods of the invention can obtain consistent results by, e.g., automated reaction timing to provide highly repeatable nucleic acid fragmentation, labeling, and/or hybridizations. For example, the methods of labeling nucleic acids can include programming a time and temperature sequence into a programmable temperature control module, fragmenting the nucleic acids with a fragmentation reaction solution in a reaction chamber of the module, inhibiting the fragmentation by raising the chamber to a reaction termination temperature, and labeling one or more nucleic acid fragments produced by the fragmentation reaction solution with a detectable marker. The programmed time and temperature sequence can provide precise control, e.g., of the inhibitory temperature increases used to terminate some reactions to provide consistent labeled probe from run to run. The methods can further include hybridization of the labeled fragments with target sequences and detection of the hybridization.

Programmable temperature control modules can include, e.g., instrumentation capable of receiving operator input of time/temperature profiles and substantially producing the profile conditions in a reaction chamber. For example, operator input can include programming by entry of time and temperature parameters into an operator interface of the module. The time and temperature programmable temperature control modules can include, e.g., resistive heating elements, refrigerants, thermoelectric devices, programmable heat blocks, programmable water baths, thermocyclers, microfluidic systems, and/or the like. Control modules can be controlled by a logic device, such as a computer. Reaction chambers can include, e.g., Eppendorf tubes, tube in a thermocycler blocks, tubes in thermocycler racks, wells in a multiwell plate, wells in a heat block, chambers in a microfluidic device, and/or the like.

Precise timing and temperature control in the programmable temperature control module can produce consistently labeled probe for sensitive comparisons between hybridization assays. In one aspect of the invention, chambers of modules can provide transitions to new temperature settings within about 1 second from the time intended for the transition. In another aspect, chamber temperatures can approach within about 1° C. of a programmed temperature, within about 15 seconds of the programmed time. In still other aspects, the chamber temperature can remain within 0.5° C. of a programmed temperature once the chamber comes within 0.5° C. of the programmed temperature.

Methods of labeling nucleic acids can include, e.g., fragmentation of the nucleic acid to a consistent average size and binding of a detectable marker to the fragments. The nucleic acids can be, e.g., genomic DNA of an individual, pooled genomic DNA from 2 or more individuals, DNA from healthy individuals, DNA from individuals presenting a disease state, alleles of a gene, single nucleotide polymorphisms, one or more mutations, one or more RNA, one or more cDNA, recombinant DNAs, a PCR product, subsequences of these nucleic acids, or compliments of these nucleic acids, and/or the like. PCR product for fragmentation and labeling can be provided, e.g., by polymerase chain reaction of genomic DNA wherein one or more PCR primers comprise sequences bracketing a single nucleotide polymorphism in the genomic DNA nucleic acid sequence. The genomic DNA can be pooled genomic DNA from two or more individuals, such as, e.g., genomic DNA from healthy individuals or genomic DNA from individuals presenting one or more disease state.

The time and temperature sequence programmed into a module for a fragmentation reaction can consecutively hold the reaction chamber at a programmed temperature ranging, e.g., from about 25° C. to about 50° C., for about 3 minutes to about 10 minutes, before said raising the fragmentation reaction to a termination temperature between about 90° C. and about 100° C. A fragmentation reaction to break the nucleic acid into consistently sized fragments can comprises, e.g., DNase I, a restriction endonuclease, a deoxyribonuclease, a ribonuclease, a glycosylase, an intercalating agent, and/or the like.

In methods of the invention, the consistently sized nucleic acid fragments can be labeled with detectable markers. For example, the nucleic acid fragments can be combined with a labeling component in a reaction solution to incorporate the detectable markers. The labeling reaction components can include, e.g., terminal transferase, alkylating agents, Klenow fragments, a DNA polymerase, and/or the like. Detectable markers for incorporation to the nucleic acids can include, e.g., fluorescent groups, fluorescein derivatives, radioactive isotopes, chromogenic compounds, and/or the like. The labeling reaction can take place in the programmable temperature control module, for example, by introducing the labeling component directly into the fragmentation reaction solution after termination of the nucleic acid fragmentation. In such a case, the nucleic acid fragments can be labeled to an extent controlled by a time and temperature sequence programmed into the temperature control module. For example, termination (inhibiting) of labeling can be precisely controlled by raising the labeling reaction chamber to a labeling termination temperature at a labeling termination time according to a sequence programmed into the module.

Labeled nucleic acid fragments can act as hybridization probes for use in the detection and/or quantitation of target nucleic acids of interest. Such target nucleic acids can be, e.g., a sequence containing one or more single nucleotide polymorphisms, a sequence or subsequence of an allele associated with a disease state, compliments of these sequences, and/or the like. The target nucleic acids can be bound to a solid support, e.g., in an array, or on a bead, a membrane, a chip, a nylon, a nitrocellulose, a plastic, a ceramic, glass, a metal, a self assembled monolayer, and/or the like. The target nucleic acids can preferably be single stranded DNA, e.g., having a nucleic acid sequence length from about 100 bases to about 10 bases. Labeled nucleic acid fragments (probes) can be combined in a hybridization solution to hybridize with nucleic acid targets on a solid support. The hybridization solution can be adjusted the to a desired hybridization temperature, e.g., in a chamber of a programmable temperature control module.

Hybridization of labeled fragments to target nucleic acid sequences can be detected and/or quantitated using techniques described herein. Labeled probe, e.g., bound to target, can be detected with a detector suitable for detection the particular marker. For example, the detector can comprise a photodiode, a photodiode array, a CCD array, a laser, a microscope, a fluorometer, a fluoroscope, a biosensor, a laser, a phosphoimager, photographic film, a spectrophotometer, a scintillation counter, a chromogenic compound, an enzyme, and or the like, known in the art. The detector can be configured to provide a quantitative detector signal associated with the amount of labeled nucleic acid fragments are hybridized to target nucleic acids at particular array locations. A logic device can receive detector signals from the detector for evaluation, storage, and the like.

Methods of the invention can provide an estimate of the frequency of certain nucleic acid sequences. For example, pooled nucleic acids comprising two or more different sequences can be provided, the pooled nucleic acids can be fragmented in a time and temperature programmable temperature control module, the nucleic acids fragmented in the temperature control module can be labeled with a detectable marker, the labeled nucleic acid fragment can be placed into a hybridization reaction with two or more target nucleic acids having one or more sequences complimentary to the labeled nucleic acid fragment, and a labeled nucleic acid fragment hybridized to at least one of the complimentary target nucleic acid sequences can be detected to provide a measure of the sequence frequency in the pooled nucleic acids.

The programmable temperature control modules, fragmentation reaction solutions, fragmentation temperature parameters, labeling components, detectable markers, and the like, for methods of sequence frequency estimation can be, e.g., as in the methods of fragmentation and labeling, discussed above. Labeling can take place in the programmable temperature control module, e.g., by adding a labeling component directly into a fragmentation reaction solution. The target nucleic acids can be bound to a solid support, e.g., in an array.

The frequency of nucleic acid sequences can be estimated for pools of genomic DNA from an individual, pooled genomic DNA from 2 or more individuals, DNA from healthy individuals, DNA from individuals presenting a disease state, alleles of a gene, single nucleotide polymorphisms, one or more mutations, one or more RNA, one or more cDNA, recombinant DNAs, a PCR product, subsequences thereof, or compliments thereof. The PCR product can be the result of a polymerase chain reaction with genomic DNA, wherein one or more PCR primers include sequences bracketing a single nucleotide polymorphism in the genomic DNA nucleic acid sequence. Target nucleic acids for estimation of sequence frequencies can be, e.g., a sequence containing one or more single nucleotide polymorphism, a sequence or subsequence of an allele associated with a disease state, or compliments of these sequences.

The present invention includes, e.g., systems for labeling nucleic acid fragments and for practicing methods of the invention. For example, a system for labeling nucleic acid fragments can include a fragmentation reaction chamber in a time and temperature programmable temperature control module. The reaction chamber can contain, e.g., a nucleic acid, and a fragmentation reaction solution which can fragment the nucleic acid to an extent controlled by a time and temperature sequence programmed into the temperature control module. The nucleic acid fragmentation can be inhibited by raising the chamber to a termination temperature at a time according to the programmed sequence. The system can include a labeling component to label the nucleic acid fragments by binding detectable markers to the nucleic acid fragments produced by the fragmentation reaction solution.

The time and temperature programmable temperature control modules of the systems can comprise, e.g., resistive heating elements, refrigerants, thermoelectric devices, programmable heat blocks, programmable water baths, thermocyclers, microfluidic systems, and/or the like. Reaction chambers of the systems can comprises Eppendorf tubes, wells in a multiwell plate, tubes in a thermocycler block, tubes in a thermocycler rack, wells in a heat block, chambers in a microfluidic device, and/or the like. The time and temperature programmable temperature control module can be capable of controlling reaction chamber temperatures at settings ranging, e.g., from about −10° C. to about 110° C. The systems can provide, e.g., a chamber temperature within about 1° C. of a programmed temperature within 15 seconds of the programmed time. The systems can provide chamber temperatures remaining within 0.5° C. of the programmed temperature after the chamber comes within 0.5° C. of the programmed temperature.

The systems can employ, e.g., programmable temperature control modules, nucleic acids for fragmentation and labeling, fragmentation reaction solutions, fragmentation temperature control capabilities, labeling components, labeling reaction parameters, detectable markers, and the like, as in the methods of fragmentation and labeling, discussed above and herein. Labeling can take place in the programmable temperature control module, e.g., by adding a labeling component directly into a fragmentation reaction solution.

Systems of the invention can further provide hardware, solutions, and detectors to practice hybridization assays. For example, the systems can include target nucleic acids, such as sequences containing one or more single nucleotide polymorphisms, sequences or subsequences of alleles associated with disease states, compliments of these sequences, and the like. The systems can include target nucleic acids bound to solid supports, such as, e.g., beads, membranes, chips, nylon, nitrocellulose, plastic, ceramic, glass, metal, self assembled monolayer, arrays, and the like. The target nucleic acids can be single stranded DNA, e.g., having a length from about 100 bases to about 10 bases. The system can include hybridization solutions, e.g., heated to a hybridization temperature in the reaction chamber and containing the labeled nucleic acid fragment.

The system of the invention can include a detector adapted to quantitatively detect the labeled nucleic acid fragments. The detector can comprise, e.g., a photodiode, a photodiode array, a CCD array, a laser, a microscope, a fluorometer, a fluoroscope, a biosensor, a phosphoimager, photographic film, a spectrophotometer, an eye, a chromogenic compound, or an enzyme. The detector can be configured to provide a quantitative detector signal associated with an array location after labeled nucleic acid fragments are hybridized to target nucleic acids in the array.

The system of the invention can include various components to facilitate operations and/or evaluation of assay results. For example, the system can include a logic device in communication with: a robotics device or microfluidic device to control transfer of the nucleic acids; the temperature control module to control a sequence of time and temperature; or, a detector of detectable markers to receive, evaluate or store detection signals. The system can include one or more subsystems, such as, e.g.: a robotics system that transfers samples or reagents, to, from, or within the system; a microfluidic device that transfers or purifies the nucleic acids; an incubator that maintains hybridization temperatures during hybridizations of the labeled nucleic acid fragments to one or more target nucleic acids; a detector adapted to quantitatively detect the labeled nucleic acid fragment; and/or, a logic device to control other subsystems, to evaluate detection signals, to evaluate system data, or to store system data.

The present invention includes systems for estimating nucleic acid sequence frequencies. Such systems can include, e.g.: a fragmentation reaction chamber in a time and temperature programmable temperature control module, a pool of nucleic acids contained in the reaction chamber; a fragmentation reaction solution in the reaction chamber to fragment the nucleic acid pool to an extent controlled by a time and temperature programmed into the temperature control module; a labeling component that binds a detectable marker to a nucleic acid fragment produced by the fragmentation reaction solution to label the nucleic acid fragment; one or more target nucleic acid sequences bound to locations on a solid support; and, a detector adapted to quantitatively detect the labeled nucleic acid fragments at the locations providing a quantitative detection signal so that the frequency of one or more nucleic acids in the pool of nucleic acids can be estimated.

The systems for estimating nucleic acid sequence frequencies can employ, e.g., programmable temperature control modules, reaction chambers, nucleic acids for fragmentation and labeling, fragmentation reaction solutions, fragmentation temperature control capabilities, labeling components, labeling reaction parameters, detectable markers, and the like, as in the systems in general for fragmentation and labeling, discussed above. Labeling can take place in the programmable temperature control module, e.g., by adding a labeling component directly into a fragmentation reaction solution, i.e., the labeling chamber and the fragmentation reaction chamber can be the same chamber. The target nucleic acids can include, e.g., sequences containing one or more single nucleotide polymorphisms, sequences or subsequences of an allele associated with a disease state, or compliments of these sequences. The detectors in the systems for estimating nucleic acid sequence frequencies can comprise fluorometers, fluoroscopes, biosensors, lasers, phosphoimagers, photographic film, spectrophotometers, chromogenic compounds, enzyme, and/or the like.

### DEFINITIONS

Unless otherwise defined herein or below in the remainder of the specification, all technical and scientific terms used herein have meanings commonly understood by those of ordinary skill in the art to which the present invention belongs.

Before describing the present invention in detail, it is to be understood that this invention is not limited to particular methods or analytical systems. It is also to be understood that the terminology used herein is often used to describe particular embodiments not intended to limit the claimed invention. As used in this specification and the appended claims, the singular forms “a”“an” and “the” include plural referents unless the content clearly dictates otherwise. Thus, for example, reference to “a component” can include a combination of two or more components; a reference to “an array” can include multiple arrays, and the like.

Unless defined otherwise, all technical and scientific terms used herein have the same meaning as commonly understood by one of ordinary skill in the art to which the invention pertains. Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

As used herein, the term “reaction chamber” refers to chambers used to contain reactions, such as fragmenting, labeling, or hybridization reactions, in the methods and/or systems of the invention. Reaction chambers can include, e.g., containers, tubes, wells, channels, microchambers, microchannels, and/or the like.

The term “time and temperature programmable temperature control module”as used herein, refers to a device capable of: receiving instructions (i.e., a programmed reaction temperature sequence) describing a desired time/temperature profile, and controlling the temperature of a reaction chamber to substantially provide the conditions defined by the profile.

The term “inhibiting”as used herein, refers to stopping a reaction ,or the rate of a reaction, so that significant production of the reaction product is terminated. In methods and systems of the invention, fragmenting and labeling reactions are often inhibited by a temperature increase to a termination temperature that removes a reactant or decreases the activity of a reaction catalyst (such as, e.g., an enzyme).

The term “PCR product”as used herein, refers to nucleic acids that are the product of a polymerization in a polymerase chain reaction.

The term “pool”as used herein, refers to a mixture of nucleic acids from two or more sources. For example, pooled Genomic DNA can be a mixture of DNA from two or more individuals. A pool of nucleic acids can include PCR products from PCR amplification of pooled substrate DNA.

The term “detectable marker”as used herein, refers to markers, bound, covalently or not, to a probe nucleic acid. The marker can be directly detectable or detectable indirectly through an association with a detectable group. For example, detectable markers can include nucleotides incorporating radioactive phosphorous, or nucleotides linked to fluorescent or luminescent moieties, such as fluorescein or rhodamine. In another example, detectable markers can be nucleotides linked to biotin, wherein the marker is indirectly detectable after association with avidin linked to a fluorescent group.

### BRIEF DESCRIPTION OF THE DRAWINGS

FIG. 1 is a graphic representation of an exemplary fragmentation reaction time and temperature sequence.

FIG. 2 Basic tiling strategy. The figure illustrates the relationship between an interrogation position (I) and a corresponding nucleotide (n) in the reference sequence, and between a probe from the first probe set and corresponding probes from second, third and fourth probe sets.

FIG. 3 is a graphic representation of differences in intra-assay probe detection for probes prepared by manual versus programmed reaction conditions.

FIG. 4 is a schematic diagram of an exemplary system for labeling nucleic acids.

FIG. 5 is a schematic diagram of a thermocycler rack useful in sample processing in the systems of the invention.

FIGS. 6A to 6E are various views of an exemplary thermocycler rack embodiment.

### DETAILED DESCRIPTION

Methods and systems of the invention provide ways to consistently fragment and label nucleic acids with detectable markers, e.g., for improved detections, quantitations, and/or comparisons in hybridization analyses. The methods of labeling nucleic acids include steps with enhanced consistency from, e.g., reduced manual sample handling and automation of reaction temperature transitions. Systems of the invention for fragmenting and labeling nucleic acid fragments can improve productivity while providing more precise quantitation of nucleic acid sequences by, e.g., providing reaction chambers with precise and consistent control of nucleic acid fragmentation and labeling reactions.

Nucleic acid labeling by methods of the invention can include, e.g., programming a fragmentation time and temperature sequence into a time and temperature programmable temperature control module, fragmenting the nucleic acid in a solution contained in a chamber of the temperature control module, inhibiting the fragmentation with a programmed raising of the chamber temperature to a termination temperature that substantially reduces the fragmenting activity in the solution, and labeling fragments of the nucleic acid with a detectable marker. The labeled nucleic acid fragments can be hybridized to complimentary target nucleic acids and quantitatively detected to determine the amount of target and/or labeled nucleic acid present. The precision of reaction time/temperature profiles obtained in the methods can enhance the precision of the quantitative detection.

In systems of the invention, e.g., nucleic acids for labeling are combined into a fragmentation reaction solution contained in a temperature control module reaction chamber, and fragmented to an extent controlled by a time/temperature sequence programmed into the temperature control module. A labeling component of the system can then bind detectable markers to the nucleic acid fragments. The labeled nucleic acid fragments can be hybridized to target nucleic acids on solid supports, quantitatively detected, and evaluated by logic devices of the system. Pools of nucleic acids from multiple sources can be evaluated by the system to determine, e.g., an estimated frequency of one or more nucleic acid sequences in the pool.

### Methods of Labeling Nucleic Acids

Nucleic acids can be labeled with detectable markers in a highly repeatable fashion, using methods described herein, to allow precise quantitation, and/or accurate comparisons of nucleic acid abundance even across multiple analyses. The methods can include, e.g., fragmenting a probe nucleic acid in a fragmentation reaction solution in a chamber of a time and temperature programmable temperature control module under preprogrammed conditions which include inhibiting the fragmentation by raising the solution to an inhibition temperature. The methods can further include, e.g., labeling the resultant nucleic acid fragments with a detectable marker, hybridizing the labeled fragments to target nucleic acid sequences, and quantitatively detecting the hybridized fragments for consistent evaluation of nucleic acid amounts or proportions.

Methods of the invention can be useful, e.g., to estimate the frequency of certain gene sequences in genomic pools from individuals with common characteristics. Comparisons of sequence frequencies can be made between groups with different characteristics to establish useful correlations between particular sequences (e.g., alleles) and the characteristics (e.g., phenotypes). Gene sequences correlated with a characteristic can be identified, e.g., as those genes with a significantly higher frequency in genomic DNA pools of individuals with the characteristic than the frequency in genomic DNA pools of individuals without the characteristic. For example, labeled nucleic acid probes, prepared by the methods of the invention from a nucleic acid with a particular allele sequence (e.g., in the region of a single nucleotide polymorphism or SNP) of a gene, can be hybridized to a genomic DNA pool of 100 individuals presenting a particular disease state and separately hybridized to a genomic DNA pool of 100 healthy individuals. The intensities of markers detected where the labeled probe hybridizes to the pooled DNAs can be used to estimate the frequency of the probe sequence in each pool. Probe sequences, e.g., with a high frequency in the disease state pool and a low frequency in the healthy pool can be correlated to the disease state. Such correlated sequences can be candidates for further investigation into the causes of the disease state. More precise estimations of gene frequencies in such pools, e.g., using the methods of this invention, can allow confident detection of real, but less common, genes causing a disease, and/or detection of significant but small differences in sequence frequencies between the DNA pools.

Programming Temperature Control Modules

Programming a time and temperature sequence into temperature control modules in the methods of labeling nucleic acids can include, e.g., input of an instruction set defining desired temperature settings for reaction chambers over a time course. The means of programming a temperature time course can depend on the particular configuration of the programmable temperature control module, as described below (and in the Systems for Labeling section).

Programmable temperature control modules can include, e.g., any hardware with a temperature controllable chamber functionally associated with a time/temperature profile instruction set. The chamber can include, e.g., an Eppendorf tube, a well in a multiwell plate, a tube in a thermocycler block, a tube in a thermocycler rack, a well in a heat block, a chamber or channel in a microfluidic device, and/or the like. The time and temperature programmable temperature control module can include, e.g., a resistive heating element, a refrigerant, a thermoelectric device, a programmable heat block, a programmable water bath, a thermocycler, a microfluidic system, and/or the like. Programmable temperature control modules useful in the methods of labeling can have temperature settings controllable in the range from about −10° C. to about 110° C., from about 0° C. to about 100° C., or from about 10° C. to about 90° C.

Programming a time and temperature sequence can be performed through a mechanical or electromechanical device, but is more typically done using a digital logic device, such as, e.g., a computer. In many cases, the programmable temperature control module can have an operator interface that allows a technician to input time and temperature sequence parameters. For example, the temperature control module can have a flat panel display and a keypad associated with a logic device to receive and display a programmed sequence. The technician can input, e.g., a time and temperature pair instruction that is to be maintained by the temperature control module until it is overridden by the next instruction in chronological order. For example, a technician can toggle through rows of time/temperature pairs on the display using arrows, number keys, and enter keys on the keypad to enter:

| TIME | TEMPERATURE |
|---|---|
| 0.25 | 4 |
| 4 | 37 |
| 10 | 95 |
| 10 | 4 |

Such an instruction set can program a fragmentation reaction time and temperature sequence wherein the reaction chamber is brought to 4° C. for 15 seconds before holding at 37° C. for 4 minutes during the fragmentation, heat up to 95° C. for 10 minutes to inhibit the fragmentation, and cool back to 4° C. to stabilize the reaction solution. The logic device can optionally be a separate computer with a communication line to the temperature control module. Programming time and temperature sequences on a computer can provide a display on a computer monitor with parameter input through the computer keyboard.

Programming allows entry of desired or intended time and temperature parameters. However, the actual time and temperature profile experienced in the reaction chamber can depend on factors, such as, e.g., the precision of the logic device commands (typically very high), the heating/cooling capacity of the temperature control module, the volume of the reaction solution, the heat conductivity of the chamber, and/or the like. Often, the programmed temperature transitions are not instantaneous, but the programmable temperature control modules of the invention can, e.g., repeatedly provide substantially identical temperature versus time profiles in reaction chambers for the consistent labeling benefits of the invention.

Fragmenting Nucleic Acids

Nucleic acids for labeling using methods described herein can be fragmented, e.g., to provide probes of a desired size (i.e., length in bases) and/or to provide additional ends for attachment of detectable markers. The nucleic acids can be fragmented according to the invention to provide, e.g., probes with good hybridization reaction kinetics, probes with consistent size, and/or probes with consistent detectable marker binding. Inconsistent fragmentation can result in poor hybridization assay sensitivity, inaccurate quantitation, imprecise quantitation, poor interassay variability, and/or the like. For example, if the nucleic acids are over fragmented, the binding sequences can be shortened, thus reducing hybridization binding under stringent conditions. If the nucleic acids are under fragmented, they can be labeled less extensively, can have slow hybridization kinetics, can be bound too tightly to target sequences that are imperfect compliments, and/or can be subject to shear removal under stringent hybridization conditions. Inconsistent fragmentation can provide probes that have different binding characteristics and/or different detectable marker intensities from batch to batch, thereby raising the statistical background noise between hybridization assays and lowering the precision of associated quantitations and comparisons.

Nucleic acids fragmented for labeling in the invention are generally nucleic acids with sequences useful as hybridization probes. Nucleic acids for labeling can include, e.g., genomic DNA of individuals, pooled genomic DNA from 2 or more individuals, DNA from healthy individuals, DNA from individuals presenting a disease state, alleles of a gene, nucleic acids with sequences having single nucleotide polymorphisms, nucleic acids with one or more mutations, RNAs, cDNAs, PCR products, and/or the like. PCR products can be, e.g., amplifications of DNA using primers bracketing sequences complimentary to target sequences of interest, such as gene allele sequences with or without SNPs. PCR products can be prepared, e.g., according to published U.S. patent application No. 20030108919, “Methods for Amplification of Nucleic Acids”to Kautzer, et al, which is incorporated by reference for all purposes.

Fragmentation of nucleic acids generally includes physical, chemical, or enzymatic breakage of nucleic acid chains into smaller fragments. Fragmenting nucleic acids for labeling according to methods of the invention can include incubation of the nucleic acids in a fragmentation reaction solution at a certain temperature for a certain amount of time. The fragmentation solution can contain fragmenting components, such as, e.g., DNase I, restriction endonucleases, deoxyribonucleases, ribonucleases, glycosylases, intercalating agents, and/or the like. In one embodiment, the fragmenting component is DNase I present in the fragmenting solution with an activity ranging from about 0.002 U/ul to about 0.003 U/ul, or about 0.0024 U/ul.

Precise temperature control in the fragmentation reaction, and precise control of the fragmentation reaction duration, are important aspects of the methods of labeling which help provide consistent preparation of labeled nucleic acid probes. Time and temperature programmable temperature control modules are generally under control of logic devices with precise timing capabilities, and/or with input connections from thermo-transducers in the environment of the reaction chamber for precise temperature control capabilities. Methods of labeling in the invention include controlling the temperature of the reaction chamber to within about 2° C. of the set temperature, within 1° C. of the set temperature, within about 0.5° C. of the set temperature, or within about 0.2° C. of the set temperature. The reaction chamber temperature can remain, e.g., within about 0.5° C. of a programmed temperature setting after the chamber comes within 0.5° C. of the programmed temperature. Reaction chambers of the methods can consistently begin a transition to a new temperature setting within about 1 second from the of the time intended for the transition. The temperature control modules for fragmentation can provide rapid temperature transitions at temperature change time points in programmed time and temperature sequence. For example, the chamber temperature can approach within about 1° C. of a programmed temperature, within 15 seconds, within about 10 seconds, or within about 5 seconds of the programmed time for the programmed temperature.

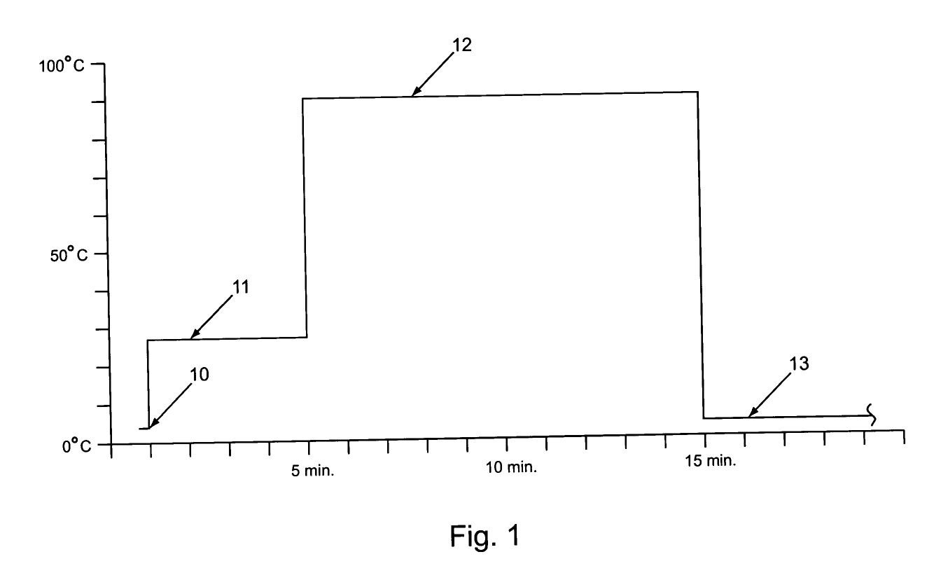

Fragmenting time and temperature sequences can include, e.g., a starting temperature and time, a fragmenting temperature and time, an inhibition temperature and time, and/or a holding temperature and time, as shown in FIG. 1. Starting temperature 10 can be a cold temperature, such as about 4° C., to stabilize the reaction solution and prevent an early start of the fragmentation reaction. The starting temperature can be consistent between fragmentation reaction runs so that, e.g., the time it takes the reaction mixture to attain the fragmenting temperature is consistent between runs. Fragmenting temperature 11 can be a temperature appropriate to the chosen fragmenting component in the reaction solution. For many enzymatic fragmenting components, a suitable temperature can range from about 30° C. to about 40° C., or about 37° C. The programmed fragmenting time can depend, e.g., on the temperature of the reaction solution, the activity of the fragmenting component, the starting size of the nucleic acid, and/or the desired average nucleic acid fragment size. Inhibition temperature 12 can be a temperature that can functionally inactivate the fragmenting component. For many enzymatic fragmenting components, a suitable inhibition temperature can range from about 80° C. to about 110° C., from about 90° C. to about 100° C., or about 95° C. Holding temperature 13 can be, e.g., a temperature low enough to stabilize the fragmentation reaction solution, and/or a temperature suitable for a subsequent labeling reaction. The hold temperature can be held for a particular length of time, or the hold temperature can be maintained indefinitely, e.g., until a technician is present to further process the fragmented nucleic acids.

Inhibiting the fragmentation can be, e.g., by raising the temperature to a point where the fragmentation component loses activity. In preferred embodiments, the fragmentation component is denatured to such an extent that no significant fragmenting activity remains, even after the solution is adjusted to a lower temperature. The timing of the inhibiting temperature onset at the inhibition temperature time point can affect fragment consistency between fragmentation runs and average fragment size. Programming the inhibition temperature time point into a time and temperature controlled programmable temperature control monitor can provide enhanced fragmentation precision. Late onset of the inhibiting temperature can result in shorter fragments and early onset of the inhibiting temperature can result in longer fragments. Nucleic acid fragments in methods of the invention can range in average size from about a 15-mer oligonucleotide to about 10,000 base pairs (bp; or bases if the nucleic acid is single stranded), from about 25 bp to about 1000 bp, or from about 50 bp to about 100 bp.

In some preferred embodiments of the methods, the programmed fragmenting time and temperature sequence can include, e.g., starting at about 0° C. to about 10° C. for about 0.1 minutes to about 1 minutes, fragmenting at about 30° C. to about 40° C. for from about 2 minutes to about 30 minutes, inhibiting at about 80° C. to about 100° C. for from about 5 minutes to about 15 minutes, and/or holding at about 0° C. to about 10° C. for about 0.5 minutes to about 10 minutes, or more. In a more preferred embodiment, the time and temperature sequence includes holding the chamber at a programmed temperature ranging from about 25° C. to about 50° C., for about 3 minutes to about 10 minutes, before said raising to the inhibition temperature of from about 90° C. to about 100° C. In a more preferred embodiment of the methods, the programmed fragmenting time and temperature sequence includes, e.g., starting at about 4° C. to for about 0.25 minutes, fragmenting at about 37° C. for about 4 minutes, inhibiting at about 95° C. for about 5 minutes, and holding at about 4° C. indefinitely.

Labeling Fragments

Nucleic acid fragments from the fragmenting reaction can be labeled with detectable markers, e.g., so that they can act as detectable probes for hybridization with complimentary target nucleic acids. Precision of labeling can be enhanced, e.g., by incubating the labeling reaction in a chamber of a temperature control module programmed with a labeling time and temperature sequence.

Nucleic acid fragments, prepared as described above, can be labeled with detectable markers by reactions with labeling components in the labeling reaction solution. Labeling components can include, e.g., marker molecules having chemically reactive linker groups, or enzymes which can catalyze addition of detectable markers to nucleic acid fragments of the labeling methods. Chemically reactive labeling components can include, e.g., alkylating molecules such as mustards that can covalently bind to nucleic acids, or metals, such as Pt that can complex to nucleic acids and cross link with marker molecules. Enzymatic labeling components can include, e.g., terminal transferase, Klenow fragment, DNA polymerases, and/or the like that can incorporate marker labeled nucleotides or nucleotide analogs into the nucleic acid fragment chains.

Markers for labeling nucleic acid fragments can be quantitatively detectable by detector devices such as, e.g., fluorometers, fluoroscopes, biosensors, scintillation counters, phosphoimagers, photographic film, spectrophotometers, and/or the like. Detector schemes can include detection by development of a chromogenic compound, e.g., in the presence of an enzyme. Detectable markers for labeling of nucleic acid fragments can comprise, e.g., fluorescent groups, fluorescein derivatives, rhodamine derivatives, antibodies, radioactive isotopes, biotin, avidin, chromogenic compounds, and/or the like.

In many embodiments of the labeling methods, labeling can be by, e.g., enzymatic incorporation of nucleotides, which have covalently bound detectable markers, onto the ends of the nucleic acid fragments. For example, terminal transferase can incorporate nucleotide triphosphates with markers from the labeling reaction solution onto the end of the fragments. In one embodiment, dUTP-biotin can be polymerized onto DNase fragmented double stranded DNA using terminal transferase enzyme to label the DNA with poly-UTP-biotin tails. In the tailing reaction, the fragmented DNA is combined with terminal transferase (labeling component), dUTP-biotin (marker), and ddUTP (a tail extension terminator). The labeling reaction is incubated in a chamber of a temperature control module (here, a thermocycler) at 37° C. for 90 minutes before denaturation (inhibiting) of the terminal transferase at 95° C. for 10 minutes to terminate the reaction. Avidin linked to a detectable group, such as a fluorescent molecule, can be added to bind to the biotin of the poly-UTP-biotin tails to complete detectable marker labeling of the nucleic acid fragment.

Consistency of the labeling reactions can be affected by precise timing of labeling termination temperature initiation and consistent heating rates to the termination temperature. Time and temperature programmable temperature control modules can provide such consistency, especially as compared to manual techniques. Furthermore, handling errors can be reduced and productivity increased by controlling the time and temperature profile of the labeling reaction in the same chamber and/or module as the fragmentation reaction.

Hybridizing Labeled Fragments to Target Nucleic Acids

Nucleic acid fragments labeled with detectable markers can be used, e.g., as nucleic acid probes for hybridization with complimentary target nucleic acids. The target nucleic acids can be bound, e.g., to solid supports, e.g., arranged in arrays.

Target nucleic acids can be from, e.g., any source, and have any sequence of interest. The target nucleic acids are generally single stranded DNA to allow complimentary pairing and binding with the labeled probe molecules. Double stranded target nucleic acids can be melted into single strands by application of heat and/or hybridization buffer constituents, such as low salt and/or formamide. The target nucleic acids can be any length, but in preferred embodiments, the target molecule or complimentary target sequence can range in length from about 1000 bases to about 8 bases, from about 100 bases to about 10 bases, or about 25 bases. The length of complimentary sequences can affect hybridization stringency, as described below. Target nucleic acids in the methods can include, e.g., nucleic acids with sequences having single nucleotide polymorphisms, nucleic acids having sequences or subsequences of gene alleles, sequences associated with a disease state, mixtures of target nucleic acids, and/or compliments of these sequences.

Target nucleic acids can be bound to solid supports, such as, e.g., a bead, a membrane, a chip, a nylon, a nitrocellulose, a plastic, a ceramic, a glass, a metal, a self assembled monolayer, and/or the like. Binding the target to a solid support can facilitate handling, concentrate the target, localize target to an identifiable position (e.g., of an array), allow unbound labeled probe to be washed away, prevent unwanted mixing of target nucleic acids in a hybridization reaction solution, and/or the like. For example, target nucleic acids can be immobilized on a nitrocellulose membrane using dot blot, slot blot, Southern blot, colony transfer blot techniques, and/or the like, well known in the art. Target nucleic acids can be immobilized in patterned arrays, e.g., on a membrane or array chip, whereon targets of known origin are present at defined array locations.

Hybridization of labeled probe nucleic acids to target nucleic acids can take place under conditions of controlled stringency. Under highly stringent hybridization conditions, a probe nucleic acid will only hybridize, e.g., to a perfectly complimentary target sequence, whereas under less stringent conditions, the probe can hybridize to targets with significant numbers of mismatched base pairs. The stringency of hybridization can be affected by the length of the complimentary sequences, the ionic strength of the hybridization solution, the hybridization temperature, the G:C content of the sequences, the presence of hybridization inhibitors (such as formamide) in the hybridization solution, and/or the like. The stringency of hybridization can be adjusted so that, e.g., as little as one uncomplimentary base in a 25 base target sequence can cause a failure of a probe to hybridize. Formulas are available to predict appropriate hybridization conditions. For example, a stringent hybridization can take place at a hybridization temperature (T_{hyb}) 20° C. below a calculated melting temperature (Tₘ) of a complimentary target/probe nucleic acid pair, that is:
T_{hyb}=Tₘ−20° C.,
or
T_{hyb}=[49° C.−(0.41×% G+C)−600/l]−20° C.,
where % G+C is the percent of guanine plus cytosine in the complimentary sequences, and 1 is the length of the complimentary sequences in bases. In many cases, the stringency of a hybridization can be fine tuned empirically to obtain the desired result, e.g., a stringency where hybridization occurs with perfectly complimentary sequences but fails if the sequences have one mismatched base pair (or a significant percentage of mismatched base pairs).

It can be convenient in the methods to prepare hybridization buffer with the probes in chambers of the temperature control module. For example, hybridization buffer constituents can be added to a completed labeling reaction in a module chamber, the chamber can be heated to the melting temperature of the probe, and the hybridization mixture can be adjusted to a desired hybridization temperature before application of the probe to target nucleic acids on a solid support. Optionally, the hybridization can take place in the chamber, e.g., by adding solid support beads with bound target to hybridization solution in the chamber for hybridization.

Hybridization to Array Chips

The invention provides a number of ways to compare a polynucleotide of known sequence (a reference sequence) with variants of that sequence (e.g., sequences of unknown sample probes prepared by fragmentation and labeling, as described above). Identification of sequence variations can be useful, e.g., in identification of genotypes associated with disease states, and/or identification of individuals most likely to benefit from drug therapies. For example, the CFTR gene and P53 gene in humans have been identified as the location of several mutations resulting in cystic fibrosis or cancer respectively. In another example, mutations or SNPs associated with faulty biotransformation enzymes can be identified which are required for detoxifying harmful environmental compounds or for appropriate metabolism of drugs. This information on sensitivities of individuals can allow customized treatments of preventive measures. The comparison can be performed at the level of entire genomes, chromosomes, genes, exons, or introns. The gene sequence and copy number can be determined with high precision and high throughput using methods of the invention to probe target arrays on a chip.

For example, arrays of oligonucleotide targets can be immobilized on a solid support for hybridization with unknown probe sequences. Nucleotide sequences of a probe at a particular position can be identified by hybridization to four target oligonucleotides in the array differing only by having A, T, G or C at the position. Quantitative comparisons can be made relative to hybridizations of reference probes of known sequence with the same targets. Sequencing and quantitative comparison methods using labeled probes of the invention in chip array hybridizations can provide high throughput analyses with reliable and consistent results.

Array chips can be designed to contain targets exhibiting complementarity to one or more selected known reference sequences. The chips can be used to read a reference sequence itself or variants of that sequence. Unknown variant probe sequences can differ from the reference sequence at one or more positions but show a high overall degree of sequence identity with the reference sequence (e.g., at least 75, 90, 95, 99, 99.9 or 99.99%). Any polynucleotide of known sequence can be selected as a reference sequence. Reference sequences of interest can include sequences known to include “normal” sequences, mutations, and/or polymorphisms associated with phenotypic changes having clinical significance in human patients.

The basic tiling strategy for an array chip can include, e.g., logically located immobilized target sequences for analysis of probe sequences having a high degree of sequence identity to one or more selected reference sequences. The strategy can be illustrated for an array that is subdivided into four target sets. A first target set can comprise a plurality of targets exhibiting perfect complementarity with a selected reference sequence. Within a segment of complementarity, each target in the first target set can have at least one interrogation position that corresponds to a nucleotide in the reference sequence. That is, the interrogation position can be aligned with the corresponding nucleotide in the reference sequence, when the target and reference sequence are aligned to maximize complementarity between the two. If a target has more than one interrogation position, each corresponds with a respective nucleotide in the reference sequence. The identity of an interrogation position and corresponding nucleotide in a particular target in the first target set cannot be determined simply by inspection of the target in the first set. However, an interrogation position and corresponding nucleotide can be defined by comparative analysis of hybridizations with the first target set to hybridizations with corresponding probes from additional target sets, as will become apparent.

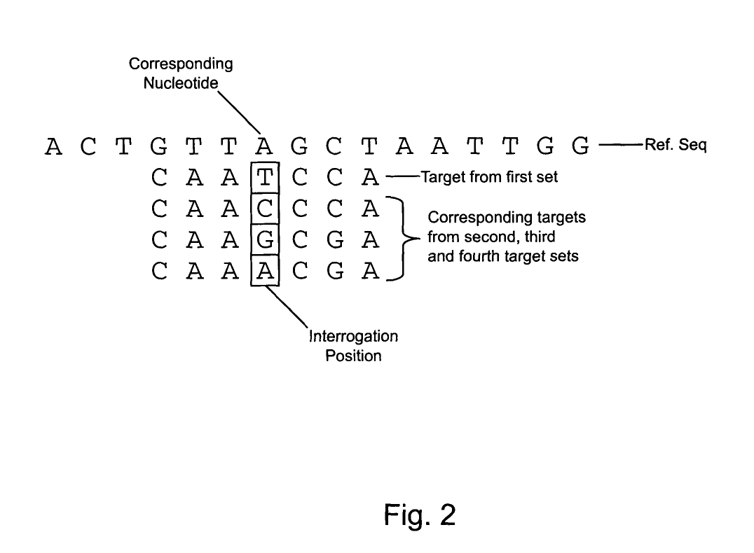

For each target in a first set, there can be, for purposes of the present illustration, up to three corresponding targets from three additional target sets, as shown in FIG. 2. Thus, there are four targets corresponding to the four possible nucleotides at the position of interest in the reference sequence. Each of the four corresponding targets has an interrogation position aligned with that nucleotide of interest. Usually, the targets from the three additional target sets are identical to the corresponding target from the first target set with one exception. The exception is that at least one (and often only one) interrogation position, which occurs in the same position in each of the four corresponding targets from the four target sets, is occupied by a different nucleotide in the four target sets. For example, for an A nucleotide in the reference sequence, the corresponding probe from the first target set has its interrogation position occupied by a T, and the corresponding targets from the other three target sets have their respective interrogation positions occupied by A, C, or G, with a different nucleotide in each target at the position of interest.

The targets can be oligodeoxyribonucleotides or oligoribonucleotides, or any modified forms of these polymers that are capable of hybridizing with a probe nucleic sequence by complementary base-pairing. Complementary base pairing means sequence-specific base pairing which includes e.g., Watson-Crick base pairing as well as other forms of base pairing such as Hoogsteen base pairing. Modified forms include 2′-O-methyl oligoribonucleotides and so-called PNAs, in which oligodeoxyribonucleotides are linked via peptide bonds rather than phophodiester bonds. The targets can be attached by any linkage to a support (e.g., 3′, 5′ or via the base). 3′ attachment is more usual as this orientation is compatible with the preferred chemistry for solid phase synthesis of oligonucleotide targets.

The number of targets in the first target set (and as a consequence the number of targets in additional target sets) depends on the length of the reference sequence, the number of nucleotides at the positions of interest in the reference sequence, and the number of interrogation positions per target. In general, each nucleotide position of interest in the reference sequence requires the same interrogation position in the four sets of targets. Consider, as an example, a reference sequence of 100 nucleotides, 50 of which are of interest, and targets each having a single interrogation position. In this situation, the first target set requires fifty targets, each having one interrogation position corresponding to a nucleotide of interest in the reference sequence. The second, third and fourth target sets each have a corresponding target for each target in the first target set, and so each also contains a total of fifty targets. The identity of each nucleotide of interest in the reference sequence can be determined by comparing the relative hybridization signals to four targets having interrogation positions corresponding to that nucleotide from the four target sets.

In some reference sequences, every nucleotide is of interest. In other reference sequences, only certain portions in which variants (e.g., mutations or polymorphisms) are concentrated are of interest. In other reference sequences, only particular mutations or polymorphisms and immediately adjacent nucleotides are of interest. Usually, the first target set has interrogation positions selected to correspond to at least a nucleotide (e.g., representing a point mutation) and one immediately adjacent nucleotide. Usually, the targets in the first set have interrogation positions corresponding, e.g., to at least 3, 10, 50, 100, 1000, or more contiguous nucleotides of the reference sequence. The targets usually have interrogation positions corresponding, e.g., to at least 5%, 50%, 90%, 99% or sometimes 100% of the nucleotides in a reference sequence. Frequently, the targets in the first target set completely span the reference sequence and overlap with one another relative to the reference sequence.

The number of targets on an array chip can be quite large (e.g., 10⁵-10⁶). However, often only a relatively small proportion (i.e., less than about 50%, 25%, 10%, 5% or 1%) of the total number of targets of a given length are selected to pursue a particular tiling strategy. For example, a complete set of octomer targets comprises 65,536 targets; thus, an array of the invention typically has fewer than 32,768 octomer targets. A complete array of decamer targets comprises 1,048,576 targets; thus, an array of the invention typically has fewer than about 500,000 decamer targets. Often arrays have a lower limit of 25, 50 or 100 targets and an upper limit of 1,000,000, 100,000, 10,000 or 1000 targets. The array chips can have other components besides the targets such as linkers attaching the targets to a support.

For conceptual simplicity, the targets in a set are usually arranged in order of the sequence in a lane across the chip. A lane can contain a series of overlapping targets, which represent or tile across, the selected reference sequence. The components of the four sets of targets are usually laid down in four parallel lanes, collectively constituting a row in the horizontal direction and a series of 4-member columns in the vertical direction. Corresponding targets from the four target sets (i.e., complementary to the same subsequence of the reference sequence) occupy a column. Each target in a lane usually differs from its predecessor in the lane by the omission of a base at one end and the inclusion of additional base at the other end. However, this orderly progression of targets can be interrupted by the inclusion of control targets or omission of targets in certain columns of the array. Such columns can serve as controls to orient the chip, or to gauge the background, which can include probe sequences nonspecifically bound to the chip.

The target sets are typically laid down in lanes such that all targets having an interrogation position occupied by an A form an A-lane, all targets having an interrogation position occupied by a C form a C-lane, all targets having an interrogation position occupied by a G form a G-lane, and all targets having an interrogation position occupied by a T (or U) form a T lane (or a U lane). The interrogation position can be anywhere in a probe but is usually at or near the central position of the probe to maximize differential hybridization signals between a perfect match and a single-base mismatch. For example, for an 11 mer probe, the central position, often used as an interrogation position, is the sixth nucleotide.

Although the array of targets is usually laid down in rows and columns as described above, such a physical arrangement of targets on the chip is not essential. Provided that the spatial location of each target in an array is known, the data from the targets can be collected and processed to yield the sequence of a target irrespective of the physical arrangement of the probes on a chip. In processing the data, the hybridization signals from the respective targets can be reasserted into any conceptual array desired for subsequent data reduction whatever the physical arrangement of targets on the chip.

In some chips, all targets are the same length. Other chips employ different groups of target sets, in which case the targets are of the same size within a group, but differ between different groups. For example, some chips have one group comprising four sets of targets, as described above, in which all the targets are 11 mers, together with a second group comprising four sets of targets in which all of the targets are 13 mers. Of course, additional groups of targets can be added. Thus, some chips contain, e.g., four groups of targets having sizes of 11 mers, 13 mers, 15 mers and 17 mers. Other chips can have different size targets within the same group of four target sets. In these chips, the targets in the first set can vary in length independently of each other. Targets in the other sets are usually the same length as the target occupying the same column from the first set. However, occasionally different lengths of targets can be included at the same column position in the four lanes. The different length targets can be included, e.g., to equalize hybridization signals from targets irrespective of whether A-T or C-G bonds are formed at the interrogation position.

The length of targets can be important in distinguishing between a perfectly matched target and targets showing a single-base mismatch with the probe sequence. The discrimination is usually greater for short targets. Shorter targets are usually also less susceptible to formation of secondary structures. However, the absolute amount of probe sequence bound, and hence the signal, is greater for larger targets. The target length representing the optimum compromise between these competing considerations can vary depending on, e.g., the GC content of a particular region of the probe DNA sequence, secondary structure, synthesis efficiency, and cross-hybridization. In some regions of the probe, depending on hybridization conditions, short targets (e.g., 11 mers) can provide information that is inaccessible from longer targets (e.g., 19 mers), and vice versa. Maximum sequence information can be read by including several groups of different sized targets on the chip as noted above. However, for many regions of the probe sequence, such a strategy provides redundant information in that the same sequence is read multiple times from the different groups of targets. Equivalent information can be obtained from a single group of different sized targets in which the sizes are selected to maximize readable sequence at particular regions of the probe sequence. The strategy of customizing target length within a single group of target sets minimizes the total number of targets required to read a particular probe sequence. This can leave ample capacity for the chip to include targets to other reference sequences.

Complimentary target sequences can be designed for either strand of the reference sequence (e.g., coding or non-coding). Some array chips can contain separate groups of targets, one complementary to the coding strand, the other complementary to the noncoding strand. Independent analysis of coding and noncoding strands can provide largely redundant (but confirmatory) information. However, the regions of ambiguity in reading the coding strand are not always the same as those in reading the noncoding strand. Thus, combination of the information from coding and noncoding strands increases the overall accuracy of sequencing.

Analysis of array chip hybridization results can reveal whether labeled probe sequences of the invention are the same or different from reference sequences. For example, if the two are the same, all targets in the first target set can show a stronger hybridization signal than the corresponding targets from other target sets. If the two are different, most targets from the first target set will still show a stronger hybridization signal than corresponding targets from the other target sets, but some targets (e.g., having a single nucleotide sequence difference between the reference and probe) from the first target set will not. Thus, when a target from other target sets light up more strongly than a corresponding probe from the first probe set, this provides a simple visual indication that the probe sequence and reference sequence differ. Furthermore, the known sequence of the other target set can indicate what nucleotide (A, T, G, or C) is replaced in the probe as compared to the reference sequence. Of the four targets in a column, only one can exhibit a perfect match to the probe sequence whereas the others usually exhibit at least a one base pair mismatch. The target exhibiting a perfect match usually produces, e.g., a substantially greater hybridization signal than the other three targets in the column and is thereby easily identified.

Detecting Hybridization

Methods of detecting hybridization of probe nucleic acids to target nucleic acids can depend on the type of solid support and/or detectable marker involved. Typically, an appropriate sensor is directed to a solid support location where probe has hybridized with target nucleic acid so that an amount of bound probe can be reflected in a detector output signal.

As described above, detectable markers can include, e.g., fluorescent groups, fluorescein derivatives, radioactive isotopes, biotin-avidin-marker conjugates, chromogenic compounds, and/or the like. Detecting fluorescent detectable markers can include, e.g., direction of appropriate excitation wavelengths onto labeled probe hybridized to target at a location on a solid support, and detecting appropriate emission wavelengths. Detection of chip arrays is typically by a two dimensional imaging device, such as a charge coupled array or a photodiode array. Detecting radioactive detectable markers can include, e.g., placing punched dot blot locations into scintillation vials and counting emissions, autoradiography, phosphoimaging, and/or the like. In a preferred embodiment, Detection of chromogenic compounds, e.g., by spectroscopy can be useful in detection of probes labeled directly or indirectly with enzymes, such as, e.g., horse radish peroxidase or alkaline phosphatase.

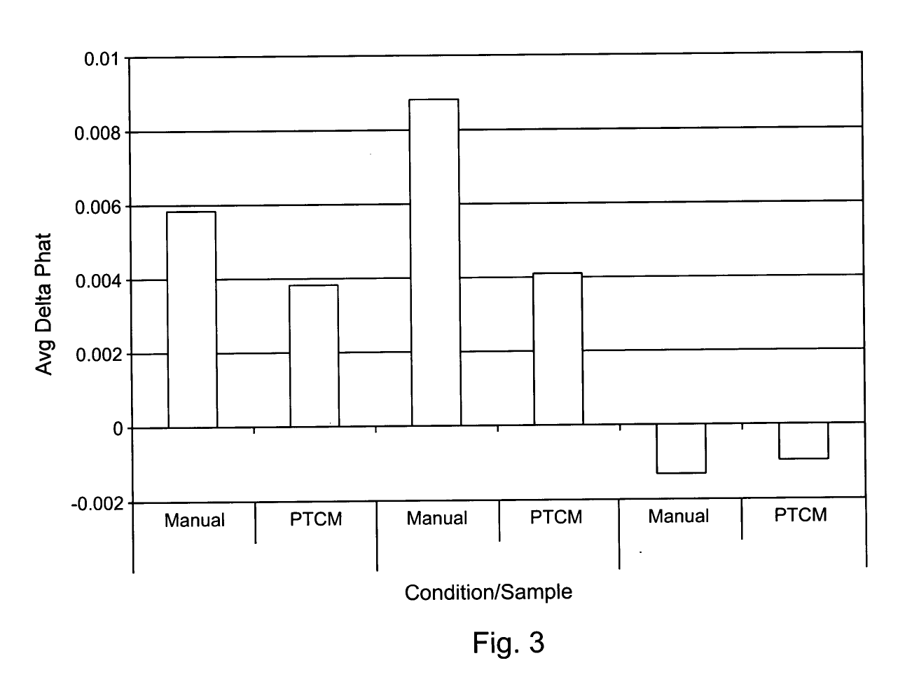

An advantage of many method embodiments for labeling nucleic acids can be, e.g., an ability to provide consistent probes for precise detection of target or probe sequence quantity. Consistent quantitative determinations within an assay and/or between assays can allow useful quantitative comparisons of target sequences of interest. The ability of the present labeling methods to provide probes with consistent detectable marker intensity or “activity” can allow evaluation of, e.g., sequence frequency estimations for nucleic acid samples, such as genomic DNA pooled from individuals with certain characteristics of interest. For example, as shown in FIG. 3, when two labeled probes were prepared according to methods of the invention, and two probes were prepared using manual techniques, quantitative results for hybridization to target sequences were about 40% more variable on the average for the manual (Manual) probes than for the automated (PTCM) probes.

In addition to detecting the presence and/or quantity of a target sequence, detecting can include, e.g., identification of a detected target sample according to a location on a solid support. For example, a target nucleic acid sample can be assigned a dot blot row/column location that can identify the target at the time of detection. In another example, target nucleic acids can have assigned locations on a microarray chip for unambiguous identification of the target during detection. In this way, e.g., detection intensity data can be associated with location/identity data for logical evaluation of assay results. These data can be received by a logic device, such as a computer, for determination of the presence, quantity, and/or proportions of hybridized probe and/or target at a location.

Systems for Labeling Nucleic Acid Fragments

Systems of the invention for labeling nucleic acid fragments can include, e.g., a time and temperature programmable temperature control module with a reaction chamber, a nucleic acid in a fragmentation reaction solution, a labeling component that can bind detectable markers to nucleic acid fragments, a solid support binding target nucleic acids, a detector that can quantitatively detect labeled probe nucleic acid fragments hybridized to target nucleic acids, and/or a logic device to control subsystems and evaluate data. The programmable temperature control module can, e.g., precisely control the fragmentation extent for the nucleic acid in the fragmentation solution to provide, e.g., probes for consistent labeling, hybridization, detection, and/or quantitation.

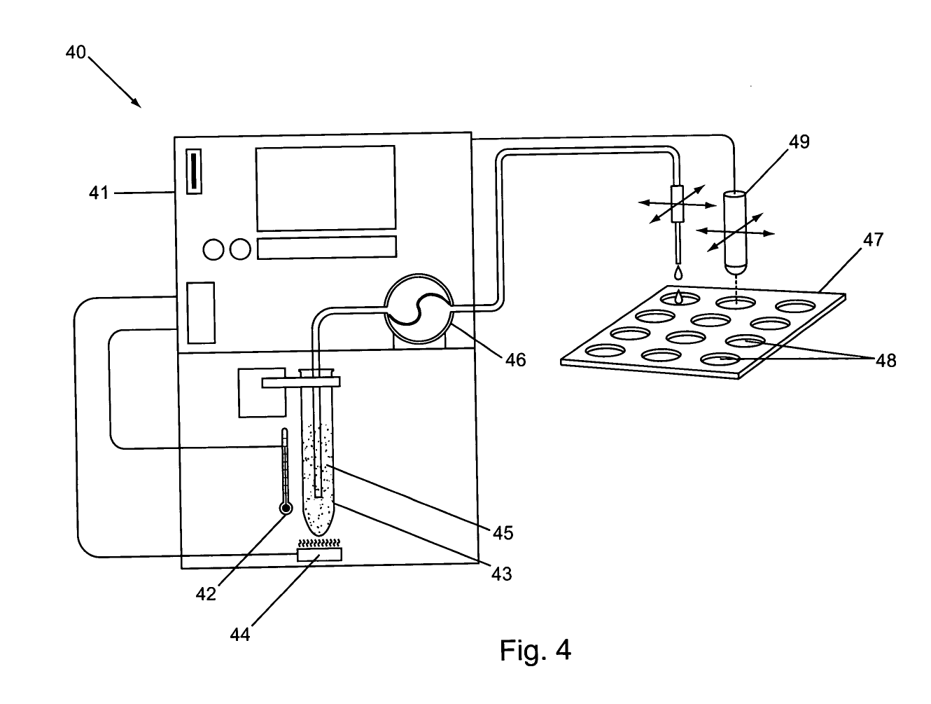

A system for labeling nucleic acid fragments as probes for quantitation of target nucleic acids (or probe sequences) can include, e.g., a logic device communicating programmable temperature control module (PTCM) temperature parameters and/or receiving marker detection signals. For example, as shown in FIG. 4, time and temperature programmable temperature control module 40 can have logic device 41 receiving temperature data from thermal transponder 42 at reaction chamber 43 and feeding back temperature setting instructions to temperature control unit 44 to provide thermostatic control and to follow a programmed temperature sequence. Reaction solution 45 can include nucleic acids and, e.g., constituents for fragmentation reactions, labeling reactions, and/or hybridization reactions. Optionally, the system can include fluid transport mechanism 46 between reaction chamber 43 and solid support 47 binding an array of target nucleic acids 48. Marker detector 49 in communication with the logic device can detect the presence, quantity, and/or location of labeled nucleic acid probes hybridized to the target nucleic acids on the solid support.

Programmable Temperature Control Modules

Programmable temperature control modules (PTCMs) of the invention can have one or more chambers that provide, e.g., precisely and/or consistently controlled time and temperature sequences. Such precision can play a significant role in obtaining uniform nucleic acid fragments, uniform labeling, and/or consistent quantitative hybridization results.

Time and temperature programmable temperature control modules of the systems can be any type known in the art. The temperature control modules can exchange heat with solutions in the chambers to provide a profile of temperature versus time substantially as programmed. The modules can be programmable with mechanical actuators associated with a mechanical time keeper, or preferably, by providing temperature/time point parameters to a digital device that communicates instructions and/or commands to an electronically settable temperature control block. Time and temperature programmable temperature control modules of the systems can include, e.g., resistive heating elements, refrigerants, thermoelectric devices, programmable heat blocks, programmable water baths, thermocyclers, microfluidic systems, and/or the like. Preferred modules can have both a thermostatically controlled heating mechanism and a cooling mechanism. For example, the module can be a computer controlled water bath with a resistive heating element and circulation to a refrigeration system evaporator coil. In a more preferred embodiment, the module has an operator interface for programming in time/temperature sequences, a thermal block for holding tubes (chambers), and a thermoelectric device capable of either heating or cooling the block. In another preferred embodiment, the module is a chamber of a microfluidic device under the control and monitoring of a computer system. Chamber temperature signals from a thermistor located near the chamber are received by the computer system which initiates heating or cooling commands to provide stable temperatures and temperature profiles at the chamber as programmed. In a most preferred embodiment of the systems, the time and temperature programmable temperature control module includes, e.g., a thermocycler.

Chambers of the systems can provide solution containment appropriate to the desired reactions. For example, the chamber can have an appropriate volume, a reaction inert surface, a thermoconductive surface, a hermetic seal to prevent evaporation, fluid channels necessary for combining or transporting reagents, and/or defined locations to identify particular reactions. The chambers of the modules can include, e.g., Eppendorf tubes, wells of a multiwell plate, tubes in a thermocycler block, wells in a heat block, chambers in a microfluidic device, tubes in a thermocycler rack, and/or the like.

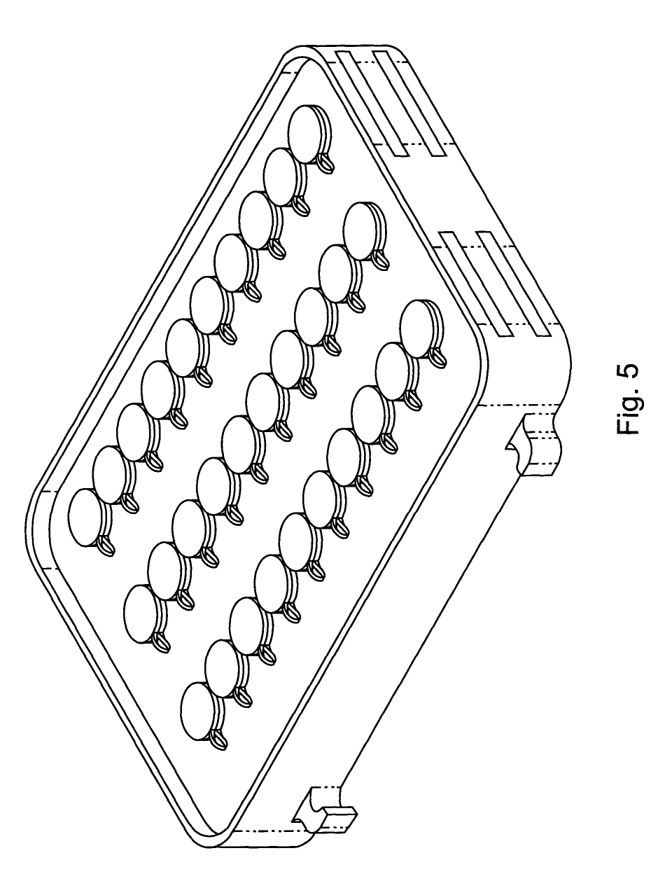

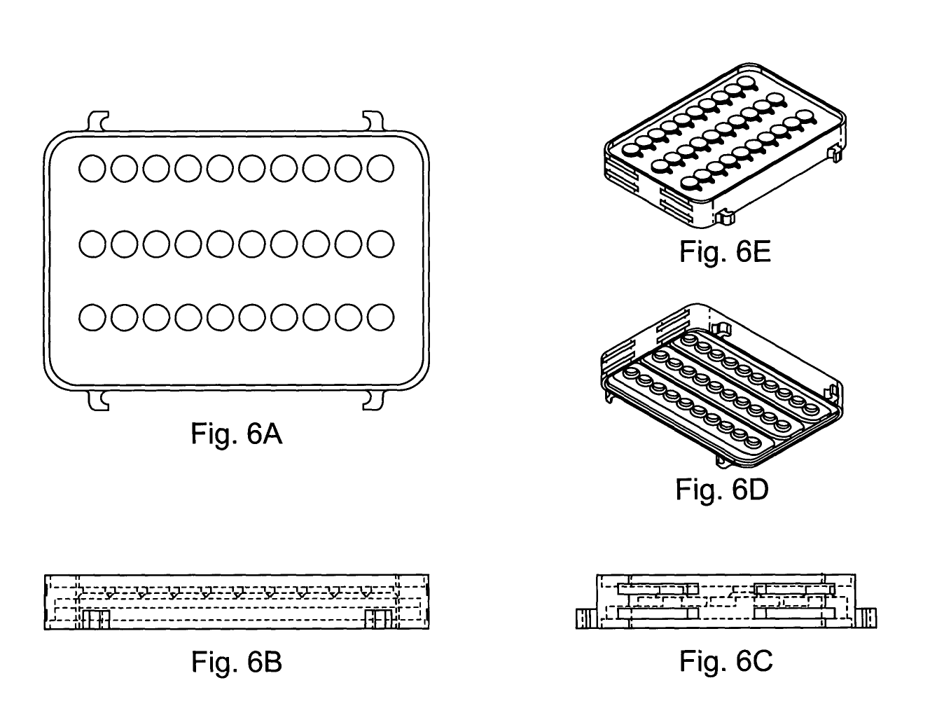

The thermocycler rack, as shown in FIGS. 5 and 6, can hold thermocycler Eppendorf tubes in positions aligned with thermocycler wells. The thermocycler rack can have an outer edge collar adapted to fit over the thermocycler block and fitting snuggly within a closed thermocycler in operation. The rack can have removable top and/or bottom seals to aid in stacking and storage. The thermocycler racks can provide the benefit of allowing sample tubes (reaction chambers) to be easily inserted and removed from a thermocycler as a unit for manipulations, such as mixing and centrifugation.

PTCMs of the systems can consistently provide desired temperature profiles at the reaction chamber, e.g., for repeatable fragmentation reactions and labeling. PTCMs can provide temperature profiles in chambers with a precision, reliability, and consistency not be obtainable by manual techniques. PTCMs can provide chamber temperatures ranging, e.g., from about −10° C. or less to about 110° C. or more, from about 0° C. to about 100° C., from about 4° C. to about 95° C., or from about 20° C. to about 90° C. The PTCMs can be programmable with time and temperature sequences having abrupt temperature changes at selected time points, and/or gradual temperature changes, such as, e.g., programmed timed temperature gradients. PTCMs can have consistent and precise thermostatic controls, e.g., wherein the chamber remains within about 0.5° C. of the program set temperature, or within about 0.2° C. of the set temperature. Once the chamber reaches a set temperature, it can remain close to the set temperature. For example, the reaction chamber temperature can remain, e.g., within about 0.5° C. of a programmed temperature setting after the chamber comes within 0.5° C. of the programmed temperature. The reaction chamber can consistently begin to transition to a new temperature setting within less than about 1 second of the time intended for the transition. The temperature control modules can provide rapid temperature transitions at temperature change time points of a programmed time and temperature sequence. For example, the chamber temperature can consistently approach within about 1° C. of a programmed temperature, within 15 seconds, within about 10 seconds, or within about 5 seconds of the programmed time for the programmed temperature. Systems of the invention can repeatedly provide temperature profiles within the ranges stated above from one run to the next. Systems with the specifications described above can be used to provide fragmented nucleic acids for labeling of nucleic acid probes at a level of consistency and precision unavailable by manual reaction temperature sequence techniques.

Fragmentation Reaction Solutions

Solutions for fragmentation of nucleic acids generally include an aqueous buffer solution with a fragmentation component that can fragment the nucleic acids to an extent controlled by the time and temperature of a fragmentation reaction. The extent of fragmentation can be controlled to provide precisely repeatable fragmentation results, e.g., by providing a consistent reaction time/temperature profile, e.g., in the reaction chamber of a PTCM.

Nucleic acids to be fragmented in a fragmentation solution can include, e.g., any nucleic acids with sequences significantly complimentary to target nucleic acids of interest. Nucleic acids for fragmentation in the systems of the invention can include, e.g., genomic DNA of an individual, pooled genomic DNA from 2 or more individuals, DNA from healthy individuals, DNA from individuals presenting a disease state, alleles of a gene, nucleic acids with sequences having a single nucleotide polymorphism, nucleic acids with mutations, RNAs, cDNAs, PCR products, recombinant DNAs, subsequences thereof, or compliments thereof, and/or the like.

Fragmentation reaction solutions can include, e.g., a fragmenting component to make internal breaks along the nucleic acid backbone resulting in a reduction of average chain length. The internal breaks can be random or can be located at sites in the nucleic acid having specific sequences. The breaks can result in blunt fragment ends, breaks in only one strand of a double stranded nucleic acid, and/or tailed ends from overlapping cuts of a double stranded nucleic acid. Fragmentation can be caused by chemical means or by enzymatic means. Fragmenting components of fragmentation reaction solutions can include, e.g., DNase I, a restriction endonuclease, a deoxyribonuclease, a ribonuclease, a glycosylase, an intercalating agent, and/or the like.

The extent of nucleic acid fragmentation in a reaction solution can be controlled by reaction conditions, such as temperature and time. At low temperatures, such as 4° C., fragmentation can be insignificant. At optimum temperatures, such as about 37° C. for many enzymes, fragmentation rates can be increased. At higher temperatures, such as 95° C., the fragmenting component can become denatured to inhibit nuclease activity. When nuclease activity is present, fragmentation can be more extensive with additional time. Systems of the invention can precisely control fragmentation time and temperature to provide, e.g., uniform fragmentation from one reaction to the next.

The extent of fragmentation can affect the degree of labeling for the fragments with detectable markers. A nucleic acid fragmented to an average smaller size will have a larger number of cut ends available for labeling by some labeling components. However, depending on the activity of the labeling component, the labeling mechanism, and/or the availability of detectable markers, the larger number of cut ends can each receive a smaller number of detectable markers. In many situations, the extent of fragmentation can affect the amount of detectable marker label bound to the nucleic acid fragments in subsequent labeling reactions. Such variable labeling can be reduced using the systems of the invention.

The extent of fragmentation can change, e.g., hybridization kinetics and/or binding affinity of probes with target nucleic acids. A probe prepared from small fragments can have faster hybridization kinetics. However, if the fragments become too small, hybridized binding can become weak and/or non-specific due to the small number of complimentary base pairs between the probe and target. On the other hand, a probe prepared from long fragments can have slower hybridization kinetics and/or have strong but less specific binding to a target under certain hybridization conditions. In certain cases, where the probe nucleic acid chain is much longer than the complimentary binding sequence, shear forces on the unhybridized chain can cause removal of the probe from the target. Accordingly, the length of fragmented nucleic acids used as probes can affect the quantity and specificity of binding to target nucleic acids. Quantitative hybridization techniques can be improved by controlling fragmentation reactions with systems of the invention.

A significant advantage of the labeling systems over manual methods can be, e.g., the ability to consistently and precisely terminate the fragmentation reaction by inhibition of the fragmenting component at high temperatures. Manual methods in the art typically require a technician to shift reaction tubes, e.g., from a fragmentation reaction water bath to a termination heat block in response to a timer alarm. Time and temperature variables are increased due to inconsistent response times to the timer alarm, tube handling order, inconsistent tube transfer times, tube handling errors, and/or the like. Systems of the invention can outperform manual methods, particularly in the precision and consistency of fragmentation reaction inhibition.

Conditions of fragmentation solutions in chambers of PTCMs can be programmed for precise and consistent control of nucleic acid fragmentation. For example, systems can consistently begin a fragmentation reaction at programmed starting temperatures between about 0° C. and about 10° C. for programmed times from about 0.1 minutes to about 10 minutes, provide active fragmentation at from about 25° C. to about 50° C. for from about 3 minutes to about 10 minutes, to inhibit fragmentation at from about 90° C. to about 100° C. for from about 2 minutes to about 20 minutes, and/or holding the inhibited reaction at from 0° C. to about 40° C. More particularly, in the case of fragmenting genomic DNA with DNase I, fragmentation conditions can be programmed to consistently provide a starting temperature of about 4° C. for about 15 seconds, transition quickly to a fragmenting temperature of about 37° C. and hold for about 4 minutes, transition quickly to inhibit fragmentation at about 95° C. for about 10 minutes, and/or hold the inhibited reaction at about 4° C. Systems of the invention can make the critical transition to the inhibition temperature with a high precision and consistency.

Labeling Components

The system of the invention includes preparation of labeled hybridization probes using labeling components to bind detectable markers to the fragmented nucleic acids. The labeling components can be added, e.g., directly to the inhibited fragmentation reaction and/or, e.g., the labeling reaction can take place in the same reaction chamber as the fragmentation. Labeling can continue at a controlled temperature for a certain period of time, e.g., as programmed into a time and temperature programmable temperature control module. Labeling can be substantially terminated, e.g., by high temperature inhibition of the labeling component.

Labeling components can bind markers, e.g., covalently through linkage chemistries and/or enzymatic reactions. Labeling component detectable markers of the systems can include, e.g., fluorescent groups, fluorescein derivatives, radioactive isotopes, chromogenic compound, and/or the like. Enzymes can be elements of labeling components, e.g., adding nucleotide analog markers to the ends of nucleic acid fragments, and/or developing colors in chromogenic detection reactions. Linker systems, such as, e.g., biotin/avidin or linker chain molecules having reactive linkage groups at one or more end, can be elements of the labeling components, e.g., to bind detectable markers or other labeling components to fragmented nucleic acid probes.

In an aspect of the invention, labeling components can be added directly to the inhibited fragmentation reaction, e.g., without requiring fragment purification steps, removal of the inhibited fragmentation reaction from the fragmentation reaction chamber, and/or exchange of buffers. For example, at the end of fragmentation inhibition, terminal transferase enzyme and nucleotide analogs having detectable marker groups can be added directly the Eppendorf tube containing the inhibited fragmentation reaction to produce a labeling reaction solution.

The labeling reaction can continue, e.g., in the same PTCM and/or same chamber as the fragmentation reaction. The precise and consistent time and temperature control of the PTCM can reduce variability in labeling from one run to the next. For example, a desirable time/temperature profile for a labeling reaction can include preincubation at from about 4° C. to about 40° C., transition to a labeling reaction at from about 25° C. to about 50° C. for from about 5 minutes to about 240 minutes, and/or transition to a termination temperature at from about 90° C. to about 100° C. for from about 2 minutes to about 30 minutes to inhibit the labeling components. In a particular case, wherein the labeling components include, e.g., terminal transferase and dUTP-biotin, the time/temperature profile for labeling can include preincubation at about 20° C. until the start of the labeling reaction transition at about 37° C. for about 90 minutes, and/or a transition to a labeling reaction termination temperature at about 95° C. for about 10 minutes. The inhibited labeling reaction can optionally be stabilized by storage at about 4° C. or less, or held at a hybridization reaction temperature of about 50° C. for receipt of prewarmed hybridization reaction components.

Solid Supports and Hybridization

Labeled nucleic acid fragments can be blended with hybridization solution components to provide conditions for hybridization with complimentary target nucleic acids bound to a solid support. The hybridization solution can be prepared in the PTCM, and/or prewarmed in the PTCM. Optionally, the target nucleic acid bound to a solid support can be added to hybridization solution in a reaction chamber for a PTCM controlled hybridization.

The hybridization solution can be prepared, e.g., in the same reaction chamber as used for the fragmentation and/or labeling reactions. Using the same PTCM and chamber can save materials, minimize handling, and/or provide the precise temperature control. Hybridization solutions can be formulated by diluting and/or adding components to the inhibited labeling reaction. For example one part 20×SSC (Na-chloride 175.32 g/L (3 M),Na3-citrate×2 H2O 88.23 g/L (0.3 M)) can be added to the inhibited labeling reaction and a quantity of water sufficient to provide the 1× volume. Other common hybridization solution components can be added, such as, e.g., deionized formamide, salmon sperm DNA, SDS, BSA, PVP, Ficoll, and/or others known in the art. Hybridization solutions, and post hybridization wash buffers, can be adjusted to affect hybridization stringency with, e.g., low ionic strength, formamide, and high temperatures reducing binding between probe and target nucleic acids.

Target nucleic acids can be bound to solid supports for hybridization with labeled nucleic acid probes. Attachment to the solid support can provide localization for direction of detector systems, identification of the target, and improved sensitivity by concentrating the target and/or probe. Solid supports of the systems can include, e.g., a bead, a membrane, a chip, a nylon, a nitrocellulose, a plastic, a ceramic, a glass, a metal, a self assembled monolayer, and or the like. In many cases, reactive groups or non-covalent interactions can hold target nucleic acids on the solid support on contact; the solid support being treated with a blocking agent before hybridization to prevent non-specific binding of probe. Optionally, target nucleic acids can be synthesized in situ on the solid support or bound to a solid support by linkage chemistries.

Target nucleic acids can be bound to solid supports in arrays, e.g., to provide identifying locations, for compatibility with the layout of detectors or mechanical fluid handling systems (such as multipipettors and robotic devices), and to provide compact target populations. Embodiments of target nucleic acids arrays include, e.g., targets on solid support beads in wells of trays, targets blotted using dot blot manifolds, targets synthesized as an array on a chip, certain bacterial colony or viral plaque blots, and the like. In a preferred embodiment, the array can be, e.g., an micro array on a silicon chip.

In order for a target and probe to hybridize into double stranded nucleic acids, they must be introduced as single strands. Some target or probe nucleic acids are single stranded to begin with, such as, e.g., some RNAs, cDNAs, many viral nucleic acids, and many synthetic nucleic acids. Double stranded target or probe nucleic acids can be melted at a temperature over their Tₘ to provide single stranded forms for hybridizations in the systems of the invention.

The length of probe and target nucleic acids can be important, e.g., to hybridization rates, specificity, and binding strength. At a given temperature, shorter probes move more quickly than longer probes and hybridize to target at a faster rate. Probes with complimentary sequences shorter than about 25 bases can be made to hybridize only to perfectly matching compliment targets by careful adjustment of the hybridization stringency, so that even a single base pair mismatch can result in no binding. Probes and targets with longer complimentary sequences generally bind more tightly. With longer complimentary sequences, highly stringent hybridization conditions can be used to prevent hybridization if there is more than, e.g., a few percent of mismatched base pairs. In the systems of labeling and hybridization of the invention, sequences complimentary between probe and target can be, e.g., from about 8 bases to about 1000 bases, from about 10 bases to about 100 bases, or about 25 bases.

Detectors

Detectors of the systems can be sensitive to the presence and/or amount of a labeled nucleic acid fragment detectable marker. The detectors can be any appropriate to the marker signature and the physical characteristics of the hybridization environment.

Detectors of the invention can detect markers known in the art. The detectors can include, e.g., fluorometers, fluoroscopes, biosensors, phosphoimagers, photographic film, spectrophotometers, eyes, and/or the like. Detectors can include photodiodes, photoarrays, CCD arrays, microscopes, lasers, LEDs, and/or other available detector components. Detectors for arrays can, e.g., sequentially analyze target locations for the presence or quantity of labeled probe. Optionally, arrays can be scanned by detectors in parallel or by an imaging system. Detectors in microfluidic systems, e.g., can include optical fibers to provide and receive detection light wavelengths to and from microchambers. Detectors can be configured to indicate locations associated with detection signals. For example, a detector mounted on X-Y sliders can sequentially scan array locations and provide X-Y coordinates of array locations along with marker detection signals. Detectors can have a communication link to transmit an analog or digital detection signal to a logic device.

Logic Devices

Logic devices of the systems for labeling nucleic acid fragments can, e.g., receive time and temperature sequences through an operator interface, communicate with temperature control modules to control time/temperature profiles, control fluid handling devices, receive detector signals, evaluate assay results, and/or store assay data.

Logic systems of the systems can include, e.g., transistors, circuit boards, integrated circuits, central processing units, computer monitors, computer systems, computer networks, and/or the like. Computer systems can include, e.g., digital computer hardware with data sets and instruction sets entered into a software system. The computer can be in communication with the detector for evaluation of the presence, identity, quantity, and/or location of a hybridized nucleic acid probe. The computer can be, e.g., a PC (Intel x86 or Pentium chip—compatible with DOS®, OS2®, WINDOWS® operating systems) a MACINTOSH®, Power PC, or SUN® work station (compatible with a LINUX or UNIX operating system) or other commercially available computer which is known to one of skill. Software for interpretation of sensor signals or monitor detection signals is available, or can easily be constructed by one of skill using a standard programming language such as Visualbasic, Fortran, Basic, Java, or the like. A computer logic system can, e.g., receive input from system operators designating time and temperature sequences, receive target and probe identification from an operator, command robotic systems to transfer the samples to the analytical system, control fluid handling systems, control sensor monitoring, receive and route sensor data, evaluate probe presence and/or analyte quantity, and/or store analytical results.

A logic device can evaluate a quantity of probe hybridized to a target, e.g., at a location on a solid support. The logic device can receive an analog detection signal containing information on the amplitude of the detection event. An analog to digital converter can provide the signal in binary form for manipulation by the logic device.

The quantity of probe detected can be determined by comparing the amplitude of the detection signal to a regression curve formula generated from detections of known (standard) samples. For example, standard samples of pooled target DNA can be provided with increasing known amounts of a target sequence of interest at locations on a solid support. Labeled nucleic acid fragment probes complimentary to the sequence can be hybridized to the standard samples, and the probe detected, to provide points on a curve of detection signal amplitude versus target sequence amount. Regression analyses, as commonly known in the art, can provide a regression curve formula representing the relationship between detection signal amplitude and the amount of target. Analysis of an unknown sample target in the system of the invention can provide a detection amplitude that can be converted to a target sequence amount based on the regression curve formula. In preferred systems, regression curves are prepared using a logic device. In some embodiments, the proportion of a target sequence in a genomic pool, such as an allele sequence, can be determined from a regression curve, e.g., of standard proportion pools to provide a measure of a sequence frequency in the unknown pool.

The ability to detect the presence of a sequence of interest over background, to quantitate a sequence of interest, to make precise interassay comparisons of samples, and to distinguish between samples with different amounts of target sequence are enhanced using methods and systems of the present invention.

### EXAMPLES

The following examples of methods and systems are offered to illustrate, but not to limit the claimed invention.

Estimation of SNP Sequence Frequency in Genomic DNA Pools

Labeled nucleic acid probes were prepared using methods and systems of the invention for quantitative hybridizations to target sequences at locations on a microarray chip. In particular, a genomic DNA pool was fragmented and labeled according to a method of the invention in time and temperature programmable temperature control modules. The resultant probe was stringently hybridized with known target nucleic acid SNP sequences at locations on a microarray chip. Frequencies of particular SNPs in the population were estimated based on the amount of probe bound at target locations of each possible SNP sequence.

A pool of PCR product was prepared by amplification of pooled genomic DNA from 300 human individuals having a common characteristic. The PCR primers chosen bracketed a region known to have a SNP in a particular gene.

The PCR product (2.7 ug) was combined in a 0.6 ml Eppendorf tube (reaction chamber) with a fragmentation solution (29.4 ul) containing DNase I (2.4 mU/ul). The fragmentation proceeded in a thermocycler type time and temperature programmable temperature control module with a time/temperature sequence of 4° C. for 15 seconds, 37° C. for 4 minutes (fragmentation), 95° C. for 10 minutes (inhibition), followed by a hold at 4° C. The Eppendorf tube was held in a thermocycler rack, shown in FIG. 5, that allows transfer, reactions, and storage of tubes without the need to individually transfer tubes between racks in separate temperature control baths and heat blocks.

The fragmented PCR product was labeled by adding 2 ul of terminal transferase (50U) directly into the inhibited fragmentation solution along with 1.5 ul of a detectable marker tailing mix (dUTP-biotin plus ddUTP). The labeling reaction proceeded in the same thermocycler as the fragmentation reaction with a time/temperature sequence of 37° C. for 90 minutes (labeling), 95° C. for 10 minutes (inhibition), followed by a hold at 4° C.

187.5 ul of a hybridization buffer was added to the inhibited labeling reactions to prepare hybridization reaction solutions. The thermocycler was programmed to provide 95° C. for 10 minutes (melting) and to hold at a 50° C. hybridization temperature. A microarray chip (solid support) having single stranded target nucleic acids at predetermined locations was warmed to the 50° C. hybridization temperature. The target nucleic acids in the array included the 4 possible SNP variants (A, T, G, C) in each of the 2 possible reading frames for a total of 8 target sequences at 8 different locations in the microarray. The target nucleic acids each presented a 25-base sequence within the region originally bracketed in the genomic pool for PCR amplification. The 50° C. hybridization reaction solution containing melted labeled PCR product fragment probes was sealed in a hybridization bag with the 50° C. microarray chip and hybridized over night in a 50° C. rotisserie incubator.

After stringent washing steps, a streptavidin-cychrom reagent (an element of the detectable marker system) was added to bind the fluorescent detectable marker to the labeled probe hybridized to complimentary target nucleic acids. The amount of probe at each target location of the array was determined by an automated detector and associated logic device. Detection signals for each location were converted to quantitative values according to a predetermined standard regression curve. The quantitative values determined for each of the 8 target locations were compared to provide a measure of the relative frequency of each SNP in the original PCR product of pooled genomic DNA at a high level of precision and confidence.

The entire process described above in this example was also undertaken using manual methods of providing the described time/temperature sequences. Technicians moved Eppendorf tube reaction chambers between racks in ice baths, 37° C. water baths, and 95° C. heat blocks to provide the sequences. With the automated and manual methods run in duplicate, the interassay variability for SNP frequency estimates were about 40% higher for the manual method than for the method of the invention.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, many of the techniques and apparatus described above can be used in various combinations.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A system for labeling one or more nucleic acid fragments, the system comprising:
a fragmentation reaction chamber in a time and temperature programmable temperature control module;
a nucleic acid in the reaction chamber;
a fragmentation reaction solution which can fragment the nucleic acid to an extent controlled by a time and temperature sequence programmed into the temperature control module; and,
a labeling component that binds detectable markers to one or more nucleic acid fragments produced by the fragmentation reaction solution, thereby labeling the nucleic acid fragments.

2. The system of claim 1, wherein the nucleic acid fragmentation is inhibited by raising the chamber to a termination temperature at a time according to the programmed sequence.

3. The system of claim 1, wherein the reaction chamber comprises: an Eppendorf tube, a well in a multiwell plate, a tube in a thermocycler block, a tube in a thermocycler rack, a well in a heat block, or a chamber in a microfluidic device.

4. The system of claim 1, wherein the time and temperature programmable temperature control module comprises, a resistive heating element, a refrigerant, a thermoelectric device, a programmable heat block, a programmable water bath, a thermocycler, or a microfluidic system.

5. The system of claim 1, wherein the time and temperature programmable temperature control module comprises temperature settings ranging from about −10° C. to about 110° C.

6. The system of claim 1, wherein the nucleic acid comprises: genomic DNA of an individual, pooled genomic DNA from 2 or more individuals, DNA from healthy individuals, DNA from individuals presenting a disease state, alleles of a gene, single nucleotide polymorphisms, one or more mutations, one or more RNA, one or more cDNA, recombinant DNAs, a PCR product, subsequences thereof, or compliments thereof.

7. The system of claim 1, wherein the fragmentation reaction solution comprises: DNase I, a restriction endonuclease, a deoxyribonuclease, a ribonuclease, a glycosylase, or an intercalating agent.

8. The system of claim 1, wherein the chamber temperature approaches within about 1° C. of a programmed temperature, within 15 seconds of a programmed time for the programmed temperature.

9. The system of claim 1, wherein a chamber temperature remains within 0.5° C. of a programmed temperature after the chamber comes within 0.5° C. of the programmed temperature.

10. The system of claim 1, wherein the labeling component comprises: an alkylating agent, a terminal transferase, a Klenow fragment, or a DNA polymerase.

11. The system of claim 1, wherein the detectable marker comprises: a fluorescent group, a fluorescein derivative, a radioactive isotope, a chromogenic compound.

12. The system of claim 1, wherein said labeling takes place in a labeling chamber of the programmable temperature control module.

13. The system of claim 12, wherein the labeling chamber and the fragmentation reaction chamber are the same chamber.

14. The system of claim 13, wherein the labeling component is added directly to the fragmentation reaction solution.

15. The system of claim 12, wherein the nucleic acid fragments are labeled to an extent controlled by a time and temperature sequence programmed into the temperature control module.

16. The system of claim 15, wherein said labeling is inhibited by raising the chamber to a labeling termination temperature at a labeling termination time according to the programmed sequence.

17. The system of claim 1, further comprising one or more target nucleic acids bound to a solid support.

18. The system of claim 17, wherein the one or more of target nucleic acids comprise an array.

19. The system of claim 17, wherein the target nucleic acids comprise single stranded DNA.

20. The system of claim 17, wherein the target nucleic acids comprise a length from about 100 bases to about 10 bases.

21. The system of claim 17, wherein the target nucleic acids comprise: a sequence containing one or more single nucleotide polymorphisms, a sequence or subsequence of an allele associated with a disease state, or compliments of these sequences.

22. The system of claim 17, wherein the solid support comprises: a bead, a membrane, a chip, a nylon, a nitrocellulose, a plastic, a ceramic, a glass, a metal, or a self assembled monolayer.

23. The system of claim 17, further comprising a hybridization solution containing the labeled nucleic acid fragment.

24. The system of claim 23, wherein the hybridization solution is heated to a hybridization temperature in the reaction chamber.

25. The system of claim 1, further comprising a detector adapted to quantitatively detect the labeled nucleic acid fragments.

26. The system of claim 25, wherein the detector comprises: a photodiode, a photodiode array, a CCD array, a laser, a microscope, a fluorometer, a fluoroscope, a biosensor, a phosphoimager, photographic film, a spectrophotometer, an eye, a chromogenic compound, or an enzyme.

27. The system of claim 25, wherein labeled nucleic acid fragments are hybridized to target nucleic acids in an array.

28. The system of claim 27, wherein the detector provides a quantitative detector signal associated with an array location.

29. The system of claim 1, further comprising a logic device in communication with: a robotics device or microfluidic device to control transfer of the nucleic acids; the temperature control module to control a sequence of time and temperature; or, a detector of detectable markers to receive, evaluate or store detection signals.

30. The system of claim 1, further comprising, one or more subsystems comprising:
a) a robotics system that transfers samples or reagents, to, from, or within the system;
b) a microfluidic device that transfers or purifies the nucleic acids;
c) an incubator that maintains hybridization temperatures during hybridizations of the labeled nucleic acid fragments to one or more target nucleic acids;
d) a detector adapted to quantitatively detect the labeled nucleic acid fragment; or,
e) a logic device to control other subsystems, to evaluate detection signals, to evaluate system data, or to store system data.

31. A system for estimating nucleic acid sequence frequencies, the system comprising:
a fragmentation reaction chamber in a time and temperature programmable temperature control module;
a pool of nucleic acids contained in the reaction chamber;
a fragmentation reaction solution in the reaction chamber, which solution fragments the nucleic acid pool to an extent controlled by a time and temperature programmed into the temperature control module;
a labeling component that binds a detectable marker to a nucleic acid fragment produced by the fragmentation reaction solution, thereby labeling the nucleic acid fragment;
one or more target nucleic acid sequences bound to locations on a solid support; and,
a detector adapted to quantitatively detect the labeled nucleic acid fragments at the locations, and to provide a quantitative detection signal;
whereby the frequency of one or more nucleic acids in the pool of nucleic acids can be estimated from the quantitative detection signal.

32. The system of claim 31, wherein the chamber comprises: an Eppendorf tube, a well in a multiwell plate, a tube in a thermocycler block, a tube in a thermocycler rack, a well in a heat block, or a chamber in a microfluidic device.

33. The system of claim 31, wherein the time and temperature programmable temperature control module comprises a resistive heating element, a refrigerant, a thermoelectric device, a programmable heat block, a programmable water bath, a thermocycler, or a microfluidic system.

34. The system of claim 31, wherein the nucleic acid comprises: genomic DNA of an individual, pooled genomic DNA from 2 or more individuals, DNA from healthy individuals, DNA from individuals presenting a disease state, alleles of a gene, single nucleotide polymorphisms, one or more mutations, one or more RNA, one or more cDNA, recombinant DNA, a PCR product, subsequences thereof, or compliments thereof.

35. The system of claim 31, wherein the fragmentation reaction solution comprises: DNase I, a restriction endonuclease, a deoxyribonuclease, a ribonuclease, a glycosylase, or an intercalating agent.

36. The system of claim 31, wherein a temperature in the chamber approaches within about 1° C. of a programmed temperature, within 15 seconds of a programmed time for the programmed temperature.

37. The system of claim 31, wherein the chamber temperature remains within 0.5° C. of a programmed temperature after the chamber comes within 0.5° C. of the programmed temperature.

38. The system of claim 31, wherein the labeling component comprises: a terminal transferase, an alkylating agent, a Klenow fragment, or a DNA polymerase.

39. The system of claim 31, wherein the detectable marker comprises: a fluorescent group, a fluorescein derivative, a radioactive isotope, a chromogenic compound.

40. The system of claim 31, wherein said labeling takes place in a labeling chamber of the programmable temperature control module.

41. The system of claim 40, wherein the labeling chamber and the fragmentation reaction chamber are the same chamber.

42. The system of claim 41, wherein the labeling component is added directly to the fragmentation reaction solution.

43. The system of claim 31, wherein the target nucleic acids comprise: a sequence containing one or more single nucleotide polymorphisms, a sequence or subsequence of an allele associated with a disease state, or compliments of these sequences.

44. The system of claim 31, wherein the detector comprises: a fluorometer, a fluoroscope, a biosensor, a laser, a phosphoimager, photographic film, a spectrophotometer, a chromogenic compound, or an enzyme.

45. A method of labeling nucleic acids, the method comprising:
programming a time and temperature sequence into a programmable temperature control module;
fragmenting the nucleic acids with a fragmentation reaction solution in a chamber of the programmable temperature control module;
inhibiting the fragmentation by raising the chamber to a termination temperature; and,
labeling one or more nucleic acid fragments produced by the fragmentation reaction solution with a detectable marker;
wherein said raising the chamber to the termination temperature is controlled by the programmed time and temperature sequence, provided by the programming.

46. The method of claim 45, wherein the time and temperature programmable temperature control module comprises: a resistive heating element, a refrigerant, a thermoelectric device, a programmable heat block, a programmable water bath, a thermocycler, or a microfluidic system.

47. The method of claim 45, wherein the reaction chamber comprises: an Eppendorf tube, a tube in a thermocycler block, a tube in a thermocycler rack, a well in a multiwell plate, a well in a heat block, or a chamber in a microfluidic device.

48. The method of claim 45, wherein the nucleic acids comprise: genomic DNA of an individual, pooled genomic DNA from 2 or more individuals, DNA from healthy individuals, DNA from individuals presenting a disease state, alleles of a gene, single nucleotide polymorphisms, one or more mutations, one or more RNA, one or more cDNA, recombinant DNAs, a PCR product, subsequences thereof, or compliments thereof.

49. The method of claim 45, wherein said programming comprises entry of time and temperature parameters into an operator interface.

50. The method of claim 45, wherein the time and temperature sequence comprises holding the chamber at a programmed temperature ranging from about 25° C. to about 50° C., for about 3 minutes to about 10 minutes, before said raising to the termination temperature of from about 90° C. to about 100° C.

51. The method of claim 45, wherein the fragmentation reaction solution comprises: DNase I, a restriction endonuclease, a deoxyribonuclease, a ribonuclease, a glycosylase, or an intercalating agent.

52. The method of claim 45, wherein the chamber temperature consistently begins to transition to a new temperature setting within about 1 second from a time intended for the transition.

53. The method of claim 45, wherein the chamber temperature approaches within about 1° C. of a programmed temperature, within 15 seconds of a programmed time for the programmed temperature.

54. The method of claim 45, wherein the chamber temperature remains within 0.5° C. of a programmed temperature after the chamber comes within 0.5° C. of the programmed temperature.

55. The method of claim 45, wherein said labeling comprises combining a labeling component with the nucleic acid fragments.

56. The method of claim 55, wherein the labeling component comprises: terminal transferase, an alkylating agent, a Klenow fragment, or a DNA polymerase.

57. The method of claim 45, wherein the detectable marker comprises: a fluorescent group, a fluorescein derivative, a radioactive isotope, or a chromogenic compound.

58. The method of claim 45, wherein said labeling takes place in the programmable temperature control module.

59. The method of claim 58, wherein said labeling comprises adding a labeling component directly into the fragmentation reaction solution.

60. The method of claim 45, wherein the nucleic acid fragments are labeled to an extent controlled by a time and temperature sequence programmed into the temperature control module.

61. The method of claim 45, further comprising inhibiting said labeling by raising the chamber to a labeling termination temperature at a labeling termination time according to the programmed sequence.

62. The method of claim 45, further comprising binding one or more target nucleic acids to a solid support.

63. The method of claim 62, wherein the one or more of target nucleic acids comprise an array.

64. The method of claim 62, wherein the target nucleic acids comprise single stranded DNA.

65. The method of claim 62, wherein the target nucleic acids comprise a nucleic acid sequence length from about 100 bases to about 10 bases.

66. The method of claim 62, wherein the target nucleic acids comprise: a sequence containing one or more single nucleotide polymorphisms, a sequence or subsequence of an allele associated with a disease state, or compliments of these sequences.

67. The method of claim 66, further comprising providing the nucleic acids for fragmentation by polymerase chain reaction of genomic DNA wherein one or more PCR primers comprise sequences bracketing a single nucleotide polymorphism in the genomic DNA nucleic acid sequence.

68. The method of claim 67, wherein the genomic DNA comprises pooled genomic DNA from two or more individuals.

69. The method of claim 68, wherein the pooled genomic DNA comprises genomic DNA from healthy individuals or genomic DNA from individuals presenting one or more disease state.

70. The method of claim 62, wherein the solid support comprises: a bead, a membrane, a chip, a nylon, a nitrocellulose, a plastic, a ceramic, a glass, a metal, or a self assembled monolayer.

71. The method of claim 62, further comprising combining the labeled nucleic acid fragment with a hybridization solution.

72. The method of claim 71, further comprising adjusting the hybridization solution to a hybridization temperature in the chamber.

73. The method of claim 45, further comprising quantitatively detecting the labeled nucleic acid fragments with a detectable marker detector.

74. The method of claim 73, wherein the detector comprises: a photodiode, a photodiode array, a CCD array, a laser, a microscope, a fluorometer, a fluoroscope, a biosensor, a laser, a phosphoimager, photographic film, a spectrophotometer, a scintillation counter, a chromogenic compound, or an enzyme.

75. The method of claim 73, wherein labeled nucleic acid fragments are hybridized to target nucleic acids in an array.

76. The method of claim 73, wherein the detector provides a quantitative detector signal associated with an array location.

77. The method of claim 45, further comprising controlling the temperature control module with a logic device, or receiving signals from a detectable marker detector with a logic device.

78. A method of providing an estimate of a frequency of a nucleic acid sequence, the method comprising:
providing pooled nucleic acids comprising two or more different sequences;
fragmenting the pooled nucleic acids in a time and temperature programmable temperature control module;
labeling a nucleic acid fragmented in the temperature control module with a detectable marker;
placing the labeled nucleic acid fragment into a hybridization reaction with two or more target nucleic acids comprising one or more sequences complimentary to the labeled nucleic acid fragment; and,
detecting a labeled nucleic acid fragment which becomes hybridized to at least one of the complimentary target nucleic acid sequences, thereby providing a measure of the sequence frequency in the pooled nucleic acids.

79. The method of claim 78, wherein the pooled nucleic acids comprise: genomic DNA of an individual, pooled genomic DNA from 2 or more individuals, DNA from healthy individuals, DNA from individuals presenting a disease state, alleles of a gene, single nucleotide polymorphisms, one or more mutations, one or more RNA, one or more cDNA, recombinant DNAs, a PCR product, subsequences thereof, or compliments thereof.

80. The method of claim 78, wherein said providing of the pooled nucleic acids comprises a polymerase chain reaction with genomic DNA, wherein one or more PCR primers comprise sequences bracketing a single nucleotide polymorphism in the genomic DNA nucleic acid sequence.

81. The method of claim 78, wherein the time and temperature programmable temperature control module comprises: a resistive heating element, a refrigerant, a thermoelectric device, a programmable heat block, a programmable water bath, a thermocycler, or a microfluidic system.

82. The method of claim 78, wherein said fragmenting comprises combining the pooled nucleic acids in a fragmentation reaction solution comprising: DNase I, a restriction endonuclease, a deoxyribonuclease, a ribonuclease, a glycosylase, or an intercalating agent.

83. The method of claim 82, further comprising adjusting a temperature of the fragmentation solution within about 1° C. of a programmed temperature, within 15 seconds of a programmed time for the programmed temperature.

84. The method of claim 78, wherein said labeling comprises combining the fragmented nucleic acid with a labeling component comprising: terminal transferase, an alkylating agent, a Klenow fragment, or a DNA polymerase.

85. The method of claim 78, wherein the detectable marker comprises: a fluorescent group, a fluorescein derivative, a radioactive isotope, or a chromogenic compound.

86. The method of claim 78, wherein said labeling takes place in the programmable temperature control module.

87. The method of claim 86, wherein said labeling comprises adding a labeling component directly into a fragmentation reaction solution.

88. The method of claim 78, further comprising binding the one or more target nucleic acids to a solid support.

89. The method of claim 88, further comprising arranging the target nucleic acids in an array.

90. The method of claim 78, wherein the target nucleic acids comprise: a sequence containing one or more single nucleotide polymorphism, a sequence or subsequence of an allele associated with a disease state, or compliments of these sequences.

91. The method of claim 78, wherein said detecting comprises detecting with a detector selected from the group consisting of: a photodiode, a photodiode array, a CCD array, a laser, a microscope, a fluorometer, a fluoroscope, a biosensor, a laser, a phosphoimager, photographic film, an eye, a spectrophotometer, a chromogenic compound, and an enzyme.

92. The method of claim 78, further comprising controlling the temperature control module with a logic device, or receiving signals from a detectable marker detector with the logic device.
